(19) United States
(12) Patent Application Publication
(10) Pub. No.: US 2004/0235733 A1
(43) Pub. Date: Nov. 25, 2004

(54) Methods and reagents for reducing polyglutamine toxicity

(76) Inventors: Joan S. Steffan, Laguna Beach, CA (US); Leslie M. Thompson, Irvine, CA (US); James Lawrence Marsh, Newport Beach, CA (US)

Correspondence Address: CHRISTIE, PARKER & HALE, LLP, P.O. BOX 7068, PASADENA, CA, 91109-7068, US

(21) Appl. No.: 10/789,518

(22) Filed: Feb. 27, 2004

Related U.S. Application Data
(60) Provisional application No. 60/451,077, filed on Feb. 27, 2003.

(51) Int. Cl. A61K 38/17

(52) U.S. Cl. 514/12

(57) ABSTRACT

Methods for treating neurological diseases and disorders using drugs that are able to block SUMOylation, enhance deSUMOylation, enhance ubiquitination, or inhibit deubiquitination, are provided.

### CROSS-REFERENCE TO RELATED APPLICATION(S)

This application claims the benefit of U.S. Provisional Application, Ser. No. 60/451,077, filed Feb. 27, 2003, the contents of which is hereby incorporated by reference in its entirety.

### BACKGROUND OF THE INVENTION

Huntington's disease (HD) is an autosomal dominant disorder which affects 1 in 10,000 individuals worldwide. It is caused by an expansion in a polyglutamine (polyQ) repeat in the amino-terminal domain of the protein, Huntingtin (Htt), a 350 kD largely cytoplasmic protein of unknown function. HD is one of several neurodegenerative diseases including spinobulbar muscular atrophy, the spinocerebellar ataxias, and DRPLA, caused by polyQ expansions in otherwise unrelated proteins.

In HD and other polyQ diseases, mutant proteins and/or pathogenic polyQ peptides produced by proteolytic processing aggregate into nuclear and/or cytosolic inclusions in neurons and in neuronal processes. These aggregates also contain other cellular proteins including transcription regulating proteins, chaperones, proteasome subunits, and ubiquitin (G. Bates, Lancet 361, 1642-4 (2003); S. Steffan, L. M. Thompson, Expert Opin. Ther. Targets 7, 201-13 (2003); A. J. Tobin, E. R. Signer, Trends Cell. Biol. 10, 531-6 (2000)).

PolyQ disease proteins can be modified in ways that change their cellular function or fate. Htt is subject to ubiquitination, which normally targets proteins for degradation (S. W. Davies et al., Cell 90, 537-548 (1997); M. A. Kalchman et al., J Biol Chem 271, 19385-94 (1996)). Mutants in ubiquitin ligases enhance polyQ toxicity in Drosophila, mouse and cell models (C. J. Cummings et al., Neuron 24, 879-92 (1999); P. Fernandez-Funez et al., Nature 408, 101-106 (2000); F. Saudou, S. Finkbeiner, D. Devys, M. Greenberg, Cell 95, 55-66 (1998)) while over expression of Parkin, an E3 ubiquitin ligase, can reduce polyQ aggregation and suppress cytotoxicity (Y. C. Tsai, P. S. Fishman, N. V. Thakor, G. A. Oyler, J Biol Chem 278, 22044-22055 (2003)). Thus, ubiquitination appears to reduce polyQ toxicity presumably by promoting degradation of toxic peptides.

SUMOylation is a post-translational modification system [for review see (F. Melchior, Annu Rev Cell Dev Biol 16, 591-626 (2000); S. Muller, C. Hoege, G. Pyrowolakis, S. Jentsch, Nat Rev Mol Cell Biol 2, 202-10 (2001))] that is biochemically similar to, but functionally distinct from ubiquitination. It involves covalent attachment of SUMO-1 (“small ubiquitin-like modifier”) to lysine residues. SUMOylation can alter protein function or subcellular location, and competition between SUMO-1 and ubiquitin for identical target lysines can protect some proteins from degradation (J. M. Desterro, M. S. Rodriguez, R. T. Hay, Mol Cell 2, 233-9 (1998); C. Hoege, B. Pfander, G. L. Moldovan, G. Pyrowolakis, S. Jentsch, Nature 419, 135-41 (2002); X. Lin, M. Liang, Y. Y. Liang, F. C. Brunicardi, X. H. Feng, J Biol Chem 278, 31043-31048 (2003)). In addition, the majority of SUMO-modified proteins found in the cell are located in the nucleus (A. Pichler, F. Melchior, Traffic 3, 381-7 (2002)) and SUMOylation can have a direct effect on nucleocytoplasmic transport.

Here we investigate the role of SUMOylation in HD pathogenesis. In this study we have found that Huntingtin is modified by SUMO-1 and co-localizes with PML and SUMO-1 in cell culture and in transgenic mouse brain. In addition, we have found that SUMO-1 modification of PML is reduced in transgenic mouse brain. Huntingtin is stabilized by SUMO-1 modification, as the same lysine residues which appear SUMOylated can also be ubiquinated. In Drosophila, we have shown that a reduction in cellular SUMOylation results in a rescue of photoreceptor neuron degeneration induced by Huntingtin. Therefore, drugs which reduce SUMO-1 modification of Huntingtin, or increase cleavage of SUMO-1 from Huntingtin, may be useful in the treatment of HD.

### BRIEF SUMMARY OF THE INVENTION

Expression of a truncated portion of the mutant Huntingtin protein encoded by exon 1 of the HD gene (Httex1p) causes disease similar to Huntington's disease (HD) in transgenic mice and flies. Httex1p can be modified by SUMO-1 and ubiquitin on lysines 6, 9, and 15; mutagenesis of these lysines to arginines reduces stability of the protein. Crossing a Drosophila model of HD with a reduced function smt3 (Drosophila SUMO) mutant results in suppression of lethality and neurodegeneration. Therefore, a drug therapy designed to lower SUMO-1 modification in the cell, or increase cleavage of SUMO-1 from target proteins, should be useful to destabilize Httex1p and block neurodegeneration in HD and other polyglutamine repeat diseases such as Kennedy's disease, dentatorubral-pallidoluysian atrophy, spinocerebellar ataxia, types 1, 2, 3 (Machado-Joseph), 6 and 7, TBP (severe cerebellar atrophy), and others, as well as other neurological diseases and disorders such as Alzheimer's disease, Parkinson's disease, amyotrophic lateral sclerosis, epilepsy, diabetes mellitus, spongiform encephalopathy, prion-related disease, and schizophrenia.

Thus, one embodiment of the present invention provides a method of treating neurodegeneration in a patient, comprising identifying a patient at risk for neurodegeneration; and administering to the patient a therapeutically effective amount of SUMOylation blocker.

Another embodiment of the present invention provides a method of treating neurodegeneration in a patient, comprising identifying a patient at risk for neurodegeneration; and administering to the patient a therapeutically effective amount of deSUMOylation enhancer.

Another embodiment of the present invention provides a method of treating neurodegeneration in a patient, comprising identifying a patient at risk for neurodegeneration; and administering to the patient a therapeutically effective amount of a Ubiquitination activator.

Another embodiment of the present invention provides a method of treating polyglutamine-expansion-related neurodegeneration in a patient, comprising identifying a patient at risk for polyglutamine-expansion-related neurodegeneration; and administering to the patient a therapeutically effective amount of SUMOylation blocker.

Another embodiment of the present invention provides a method of treating polyglutamine-expansion-related neurodegeneration in a patient, comprising identifying a patient at risk for polyglutamine-expansion-related neurodegeneration; and administering to the patient a therapeutically effective amount of deSUMOylation enhancer.

Another embodiment of the present invention provides a method of treating polyglutamine-expansion-related neurodegeneration in a patient, comprising identifying a patient at risk for polyglutamine-expansion-related neurodegeneration; and administering to the patient a therapeutically effective amount of a Ubiquitination activator.

Another embodiment of the present invention provides a method of treating a neurodegenerative disease in a patient, comprising administering to the patient a therapeutically effective amount of SUMOylation blocker.

Another embodiment of the present invention provides a method of treating a neurodegenerative disease in a patient, comprising administering to the patient a therapeutically effective amount of deSUMOylation enhancer.

Another embodiment of the present invention provides a method of treating a neurodegenerative disease in a patient, comprising administering to the patient a therapeutically effective amount of a Ubiquitination activator.

Another embodiment of the present invention provides a method of treating Huntington's disease in a patient, comprising administering to the patient a therapeutically effective amount of a SUMOylation blocker.

Another embodiment of the present invention provides a method of treating Huntington's disease in a patient, comprising administering to the patient a therapeutically effective amount of a deSUMOylation enhancer.

Another embodiment of the present invention provides a method of treating Huntington's disease in a patient, comprising administering to the patient a therapeutically effective amount of a Ubiquitination activator.

Another embodiment of the present invention provides a method of treating Kennedy's disease in a patient, comprising administering to the patient a therapeutically effective amount of a SUMOylation blocker.

Another embodiment of the present invention provides a method of treating Kennedy's disease in a patient, comprising administering to the patient a therapeutically effective amount of a deSUMOylation enhancer.

Another embodiment of the present invention provides a method of treating Kennedy's disease in a patient, comprising administering to the patient a therapeutically effective amount of a Ubiquitination activator.

Another embodiment of the present invention provides a method of treating spinocerebellar ataxia in a patient, comprising administering to the patient a therapeutically effective amount of a SUMOylation blocker.

Another embodiment of the present invention provides a method of treating spinocerebellar ataxia in a patient, comprising administering to the patient a therapeutically effective amount of a deSUMOylation enhancer.

Another embodiment of the present invention provides a method of treating spinocerebellar ataxia in a patient, comprising administering to the patient a therapeutically effective amount of a Ubiquitination activator.

Another embodiment of the present invention provides a method of treating dentatorubral-pallidoluysian atrophy in a patient, comprising administering to the patient a therapeutically effective amount of a SUMOylation blocker.

Another embodiment of the present invention provides a method of treating dentatorubral-pallidoluysian atrophy in a patient, comprising administering to the patient a therapeutically effective amount of a deSUMOylation enhancer.

Another embodiment of the present invention provides a method of treating dentatorubral-pallidoluysian atrophy in a patient, comprising administering to the patient a therapeutically effective amount of a Ubiquitination activator.

Another embodiment of the present invention provides a method of treating protein-aggregation-related neurodegeneration in a patient, comprising administering to the patient a therapeutically effective amount of a SUMOylation blocker.

Another embodiment of the present invention provides a method of treating protein-aggregation-related neurodegeneration in a patient, comprising administering to the patient a therapeutically effective amount of a deSUMOylation enhancer.

Another embodiment of the present invention provides a method of treating protein-aggregation-related neurodegeneration in a patient, comprising administering to the patient a therapeutically effective amount of a Ubiquitination activator.

Another embodiment of the present invention provides a method of treating Machado-Joseph's disease in a patient, comprising administering to the patient a therapeutically effective amount of a SUMOylation blocker.

Another embodiment of the present invention provides a method of treating Machado-Joseph's disease in a patient, comprising administering to the patient a therapeutically effective amount of a deSUMOylation enhancer.

Another embodiment of the present invention provides a method of treating Machado-Joseph's disease in a patient, comprising administering to the patient a therapeutically effective amount of a Ubiquitination activator.

### BRIEF DESCRIPTION OF THE DRAWINGS

FIG. 1. Huntingtin can be modified by SUMO-1 or Ubiquitin and co-localizes with SUMO-1 in cell culture. (A) The sequence of the Httex1p fragment with the extent of the two transgene constructs indicated. (B) SUMO-1 co-localizes with expanded Httex1p at the nuclear periphery and in inclusions of immortalized striatal neurons (cytoplasm not visible due to fixation technique used). (C) Httex1p with and without the proline-rich region is modified by HIS-SUMO-1 or by HIS-Ubiquitin in HeLa cells. Cells were co-tranfected with plasmids expressing HIS-tagged SUMO-1 or ubiquitin, and Httex1p 97QP or 103Q. The tagged protein was enriched using Ni-NTA magnetic nickel columns and detected by Western analysis using anti-Htt antibody. Mutation of all lysine residues [K] to arginine [R] (K6,9,15R) inhibits both SUMOylation and ubiquitination of Httex1p. 10% of the initial Ni-NTA lysate was TCA precipitated and subjected to Western analysis, showing relative levels of modified and unmodified Httex1p (WC-TCA). (D) Unexpanded 25QP Httex1p can be modified by SUMO-1 and ubiquitin. 97QP-GFP, 103Q-GFP, and 25QP-GFP with wt lysines 6, 9, and 15 (control) or with these lysines mutated to arginine (K6,9,15R) were transiently co-transfected with vector control, HIS-SUMO-1, or HIS-Ubiquitin. Ni-NTA enrichment was performed as in C. Unique SUMO-1 and ubiquitin-modified bands, present in the control, but not in the K6,9,15R mutants are denoted by black circles. (E) Mutation of the lysine residues singly and in combination significantly reduced or eliminated Httex1p modification by HIS-SUMO-1 or HIS-Ubiquitin in HeLa cells as demonstrated by Ni-NTA enrichment and Western analysis using anti-Htt antibodies.

FIG. 2. Huntingtin is stabilized by SUMO-1 modification and fusion of SUMO-1 to Httex1p or deletion of the proline-rich region of Httex1p decreases inclusion formation. (A) The abundance of untagged 97QP or 103Q Httex1p is reduced by mutation of amino-terminal lysines to arginines. HeLa cells were co-transfected with plasmids expressing modified Httex1p constructs and exogenous HIS-SUMO-1. Whole cell extracts were assessed by Western analysis using anti-Htt antibody. (B) Western analysis with anti-Htt antibody of SUMO-1 fused in frame with the K6,9,15R triple mutants of 97QP or 103Q in immortalized striatal cell extract demonstrates that “permanent SUMOylation” dramatically increases protein levels. Arrows indicate the unmodified size of the Htt transgenes, 97QP and 103Q. (C) Immortalized striatal cells were transiently transfected with a plasmid containing the CMV promoter fused to the gene for β^{˜} galactosidase (pCMX-βgal) along with either pcDNA3.1 vector control, 97QP, 97QP K6,9,15R, SUMO-97QP, or SUMO-97QP K6,9,15R. SUMO-97QP dramatically represses transcription of the CMV promoter. (D) Immunofluorescence analysis shows that while inclusions are observed for 97QP and its triple mutant in immortalized striatal cells, inclusion formation is reduced by SUMO-1 fusion to the 97QP and 97QP K6,9,15R. Fusion of SUMO-1 to Htt 97QP produces diffuse staining predominantly in the cytoplasm in cells where expression is visible. (E) Immunofluorescence shows that inclusions are found in immortalized striatal cells expressing 97QP but not 103Q, demonstrating a role for the proline-rich region of Httex1p in aggregation.

FIG. 3. Permanently SUMOylated Httex1p dramatically represses transcription. Luciferase assays were performed using immortalized striatal cells transiently co-transfected with either the MDR1-luciferase or WAF1-pGL3-luciferase reporters and with pcDNA3.1 vector control, 97QP, or SUMO permanently fused to 97QP (SUMO-97QP).

FIG. 4. Amino acids 1-17 of Htt can target a nuclear-localized reporter protein to the cytoplasm, independent of CRM-1 function. Leptomycin B abolishes nuclear export that is mediated by the CRM-1 export receptor. NIH-3T3 cells were transfected with the indicated GFP reporter constructs. After 24 hours cells were treated with Leptomycin B for 2 hours (+LMB) or not (Mock) and GFP localization was recorded by confocal microscopy. Single sections are shown. GFP alone locates to both cytoplasm and nucleus. When fused to a NLS, GFP is nuclear. Fusion of the first 17 amino acids of Htt to the NLS-GFP construct drives GFP to the cytoplasm and this localization is unaffected by LMB. Control experiments demonstrate that CRM dependent nuclear export signals, NES-GFP, are quite sensitive to LMB.

FIG. 5. Genetic reduction of SUMO activity in Drosophila reduces neurodegeneration in an HD fly model. (A) Neuropathology improves when SUMO levels are reduced. Flies expressing Httex1p Q93 ubiquitously in the nervous system under the control of the elav-GAL4 driver show extensive loss of photoreceptors (gray bars). Normal flies show 7 rhabdomeres in every ommatidium and the more extensive the degeneration, the fewer the number of rhabdomeres. When the level of SUMO activity is reduced by 50% in heterozyotes of the single SUMO gene in Drosophila (smt3/+), photoreceptor loss is dramatically reduced (black bars); thus, genetic reduction of SUMO activity rescues HttQ93 mediated neuropathology. T-test of significance=P<0.001. (B) Neuropathology is only modestly increased when ubiquitin activity is reduced. When the level of ubiquitin activity is reduced by 50% in heterozyotes of the Ubi63E ubiquitin gene (Ubi63E/+), photoreceptor loss is modestly more severe (black bars) than in controls expressing Htt Q93 in a normal background. Indeed the increase in severity is barely significant statistically P<0.060. Thus, genetic reduction of ubiquitin activity only slightly exacerbates HttQ93 mediated neuropathology. (C) Cytotoxicity is severely reduced by mutation of the three lysines in Htt 97QP. Transgenic Drosophila expressing Httex1p 97QP or Httex1p 97QP K6,9,15R under the control of elav-GAL4 at 27° were analyzed. Expression of the unmodified 97QP transgene produces significant photoreceptor loss (black bars). In contrast, when the transgene with the three lysines mutated is expressed, photoreceptor loss is dramatically reduced [P<0.013] indicating a strong attenuation of cytotoxicity by mutation of these lysines. D. Transgenic Drosophila expressing Httex1p 97QP or Httex1p 97QP K6,9,15R under the control of gmr-GAL4 at 27° were analyzed. Expression of the unmodified Httex1p 97QP transgene produces a visible rough eye phenotype with necrotic lesions indicative of cytotoxicity. In contrast, expression of the transgene with the three lysines mutated produces almost no detectable phenotype under the same conditions again confirming a strong attenuation of cytotoxici-ty by mutation of these lysines.

### DETAILED DESCRIPTION OF THE INVENTION

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the cell lines, constructs, and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

Long repeats of polyglutamines within specific disease genes are responsible for at least eight human neurodegenerative diseases, including Huntington's disease (HD). Expression of a truncated portion of the mutant Huntingtin protein encoded by exon 1 of the HD gene (Httex1p) causes neurodegenerative disease similar to HD in transgenic mice and flies. SUMO-1 (small ubiquitin-related modifier-1) modification of proteins affects their stability, protein-protein interactions, and/or subcellular localization. Httex1p was found to be SUMO-1 modified and ubiquitinated. Mutation of three lysine residues in the amino-terminal 17 amino acids of expanded Httex1p to arginine (K6R, K9R, and Kl5R) reduces stability of this polypeptide in cell culture. In mutagenesis studies, lysines 6, 9, and 15 were found to be important for SUMOylation of Httex1p and ubiquitin and SUMO-1 may compete for lysines 6 and 9. PML SUMOylation is reduced in HD transgenic mouse brain. Since in polyQ disease brains, nuclear body morphology is changed, a reduction in SUMOylation of PML may be instrumental in the disruption of nuclear body structure. Expanded polyQ Httex1p co-localizes with PML and SUMO-1 in nuclear bodies in transgenic mouse brain and in cell culture, consistent with many nuclear body proteins found to be modified by SUMO-1. Crossing a Drosophila model of HD with a reduced function smt3 (Drosophila SUMO) mutant results in suppression of lethality and of neurodegeneration of photoreceptor neurons in the eye. Therefore a drug therapy designed to lower SUMOylation in the cell, or increase cleavage of SUMO-1 from target proteins such as Htt, should be useful to destabilize Httex1p and block neurodegeneration in HD and other polyQ diseases.

There are currently no tested compounds that rescue the neurodegeneration or prevent progression of disease in HD patients. Blockage of the process of SUMOylation of proteins, or enhancement of the process of deSUMOylation of proteins, may prevent neurodegeneration and death caused by polyglutamine repeat diseases and other diseases caused by aberrant aggregation of protein. To our knowledge, drugs which can accomplish either of these processes have not been developed and are not available. Drugs that are able to block SUMOylation (hereinafter, “SUMOylation blocker”) or enhance deSUMOylation (hereinafter, “deSUMOylation enhancer”) should be useful in treatment of neurodegenerative disease. Potential therapeutic drugs include agents that decrease the activities of E1 SUMO activating enzyme, E2 SUMO conjugating enzyme, or E3 SUMO ligating enzyme, or which increase the activity of SUMO isopeptidase, as well as any other agent shown in be effective in reducing SUMOylation of Huntingtin and other poly-Q repeat proteins. PIAS proteins, a class of SUMO E3 ligase enzymes (a protein inhibitor of activated STAT) SUMOylate Huntingtin (data not shown) and inhibition of the function of these PIAS proteins in particular may represent a good therapeutic agent for treatment of neurodegenerative diseases. Other potential therapeutic agents include drugs designed to enhance the process of Ubiquitination (activate E1 Ubiquitin activating enzyme, activate E2 Ubiquitin conjugating enzyme, activate E3 Ubiquitin ligating enzymes; hereinafter, “Ubiquitination activators”) or to inhibit deubiquitination (such as inhibitors of Ubiquitin isopeptidase, to decrease cleavage of Ubiquitin from proteins; hereinafter “deubiquitination inhibitors”).

The present inventors have provided a clear demonstration of reduced polyglutamine toxicity in response to a loss in the ability of Drosophila cells to sumoylate proteins in vivo. It is proposed that a reduction in the SUMOylation of pathogenic mutant polyglutamine repeat protein causes it to become unstable and to be degraded much more quickly than when it is SuMOylated. Therefore, drugs designed to block SUMOylation of mutant polyglutamine repeat proteins, or drugs designed to enhance the removal of SUMO-1 from these proteins, should have a therapeutic effect in the treatment of HD and other polyglutamine-repeat diseases.

The consequence of polyglutamine repeat disease is slow and wasting death with no treatment options available. Any option to slow or prevent the process would be desirable. The invention has clear public and commercial use in the treatment of HD and other polyglutamine repeat diseases and potentially as well in neurodegenerative and psychiatric diseases in general.

### MATERIALS AND METHODS

Plasmid constructs. Httex1p-GFP fusion proteins were created by placing alternating CAG/CAA repeats, coding for either a normal range or expanded polyglutamine tract, into the context of either a truncated [first 17 amino acids plus poly(Q) repeat] or complete Huntingtin exon 1 containing the proline-rich region and subcloning into pcDNA 3.1. Untagged Httex1p constructs were created from these GFP-tagged constructs by blunting the BamHI and XbaI sites surrounding the GFP cDNA and reclosing the vector. K6R, K9R, and K15R mutations in Httex1p were created through use of double-stranded oligonucleotides containing HindIII compatible ends, encoding the first 17 amino acids of Huntingtin (plus and minus lysine to arginine mutations in amino acids 6, 9, and 15), which were ligated between the HindIII site of pcDNA3.1 in the polylinker, and the HindIII site in exon I, immediately 5′ to the CAG repeat. Double-stranded oligonucleotides were used to fuse the SV40 nuclear localization signal (MGPKKKRK) to the amino-terminus of EGFP, and Htt amino acids 1-17 were then fused to the amino-terminus of NLS-EGFP to create both NLS-EGFP and 1-17 NLS-EGFP constructs. pEGFP-Nl (BD Biosciences/Clontech) was used as a control plasmid for EGFP expression. pCMX-betagal (containing the CMV promoter fused to the beta-galactosidase gene) was the gift of B. Blumberg (UCI)/M. Tabb (UCI)/R. Evans (Salk Institute). A PCR fragment encoding amino acids 1-96 of SUMO-1 (lacking glycine 97 creating a SUMO-1 that is not susceptible to proteolytic removal by isopeptidases) was fused in frame to the amino-terminus of 97QP, 97QP K6,9,15R or 103Q K6,9,15R with SalI/NcoI linkers, creating “permanently” SUMOylated Httex1p in pcDNA3.1. pHA-SUMO-1, pHIS-SUMO-1, and pHIS-Ubiquitin were obtained from M. Nevels (Princeton), A. Dejean (Institut Pasteur)/G. David (Harvard)/R. DePinho (Harvard), and D. Bohmann (University of Rochester)/G. David (Harvard)/R. DePinho (Harvard), respectively. Synthetic oligonucleotides were used to create NES-GFP as a derivative of C2-EGFP from Clontech with the NES of PKI fused in frame at the C-terminus (GFP-NESPKI). 97QP and 97QP K6,9,15R KpnI/BamHI fragments were cloned between the EcoRI/NotI sites of pUAST to create PUAST-97QP and pUAST-97QP K6,9,15R. The proteins encoded by these constructs contain full Htt exon 1 including the DNA encoding the proline-rich region, followed by the following amino acid sequence: GSTSSRAAAARGYL. The MDR1-luciferase (a kind gift of E. Stanbridge, UCI) and WAF1-pGL3-luciferase reporter constructs were used as previously described (30).

Assays. Luciferase assays were conducted as previously reported (J. S. Steffan et al., Proc Natl Acad Sci U S A 97, 6763-8 (2000)). Magnetic nickel column Ni-NTA assays for SUMOylation and Ubiquitination were performed as previously described (S. Muller et al., J Biol Chem 275, 13321-9 (2000)). Beta-galactosidase activity was measured using the technique of Miller (J. H. Miller, Experiments in molecular-genetics, 352-355 (1972)). Briefly, 6 well dishes of HeLa cells were each transiently transfected with 100 ng pCMX-betaga1 plasmid plus/minus 100 ng test plasmid two days before harvest. The cells were lysed after a PBS wash in 200 microliters of Pharmingin 1X lysis buffer, spun in the microfuge for 30 seconds to remove debris, protein concentrations determined by Bradford assays. Beta-galactosidase activity is reported as % activity/mg protein.

Western analysis to examine levels of Huntingtin expression was done using whole cell lysates from HeLa or immortalized striatal cells broken and sonicated in Buffer A: lOmM Tris-HCl pH 7.6, 1 mM EDTA, 400 mM NaCl, 10% glycerol, 0.5% NP-40, protease inhibitors: PMSF (1 mM), Aprotenin (10 micrograms/ml), Leupeptin (10 micrograms/ml), Iodoacetamide (2 mM), and N-ethyl maleimide (20 mM). Rabbit polyclonal Anti-Htt antibody CAG 53b was obtained from Erich Wanker.

The Leptomycin B Nuclear Export Signal Assay used NIH-3T3 cells grown on glass coverslips (Fisher) and transfected using Lipofectamine 2000 according to the manufacturers protocol. Cells 24 hrs post-transfection were treated with 200 nM Leptomycin B (Sigma) or DMSO (Mock) for 2 hrs. Cells were fixed with 3% PFA and mounted using ProLong (Molecular Probes) mounting medium. Confocal microscopy was performed on a Zeiss LSM 510 confocal system using an Axiovert 100 M microscope and a Plan/Apochromat 100×/Oil objective.

Drosophila genetics. Elav>Httexon1Q93 animals were generated by crossing elav-GAL4; Sb/TM6 virgins to UAS-exon1Q93 (line#P463) homozygous males at 18° C. Exon1Q93 expressing males were then crossed to P(ry^{+t7.2=PZ})smt³⁰⁴⁴⁹³ cn¹/CyO; rY⁵⁰⁶ (Bloomington stock number 11378) females at 25° C, or to Df(3L)119/TM6,Hu ca, a deficiency that deletes the Ubi63E ubiquitin gene, (Stock #3649) and the elav>exon1Q93; smt3/+or Ubi63E/+progeny collected. Control animals were the elav>exon1Q93; CyO siblings. The eyes were scored using the pseudopupil technique (J. S. Steffan et al., Nature 413, 739-43 (2001)) at seven days post-eclosion. All crosses were performed using standard Drosophila medium.

Transgenic flies containing uas-Httex1p 97QP and uas-Httex1p 97QP K6,9,15R were generated by p-element transformation and confirmed by DNA sequencing. These constructs were crossed to flies expressing the yeast Ga14 transcriptional activator driven by either the gmr promoter which drives expression in all cells of the eye including neurons and surrounding and supporting cells (w*; P[w+mC=GAL4-ninaE.GMR]12) (M. C. Ellis, E. M. O'Neill, G. M. Rubin, Development 119, 855-65 (1993)) or the elav promoter, which drives expression in all neurons from embryogenesis on pP[GAL4-elav.L]. Since the UAS/GAL4 system is highly sensitive to temperature, cultures were grown at 25°, 27° and 29° C.

Cell culture and transfections. HeLa cells were grown in Dulbecco's modified Eagle's medium (Invitrogen) with 10% fetal bovine serum (Invitrogen). ST12.7 rat striatal cells (S. Sipione et al., Hum Mol Genet 11, 1953-65 (2002)) were grown in Dulbecco's modified Eagle's medium (Invitrogen) with 10% fetal bovine serum (Invitrogen) at 33° C. HeLa cells were transfected with Effectene (QIAGEN) and rat striatal cells were transfected with FuGENE 6 (Roche) according to the manufacturer's instructions.

Primary Antibodies. The source and working dilution of primary antibodies were follows: S830 (1:500) is a sheep polyclonal raised against a GST-exon 1 huntingtin fusion protein carrying 53Q (a kind gift from Dr. G. Bates, London United Kingdom (D. L. Smith et al., Neurobiol Dis 8, 1017-26 (2001))). CAG53b (1:5000) is a rabbit polyclonal (raised against amino acids 1-118 with 51 polyglutamines; a kind gift from Dr. E. Wanker, Berlin, Germany) anti-huntingtin antibody (S. W. Davies et al., Cell 90, 537-548 (1997)). mEM48 (1:200) is a mouse monoclonal that was raised against a GST fusion protein containing the first 256 amino acids of human huntingtin with the deletion of the polyglutamine tract (Chemicon). SUMO-1 (FL-101) (1:500) is a rabbit polyclonal and SUMO-1 (D-11)(1:200) is a mouse monoclonal, both raised against human SUMO-1 (amino acids 1-101)(Santa Cruz Biotechnology, Inc).

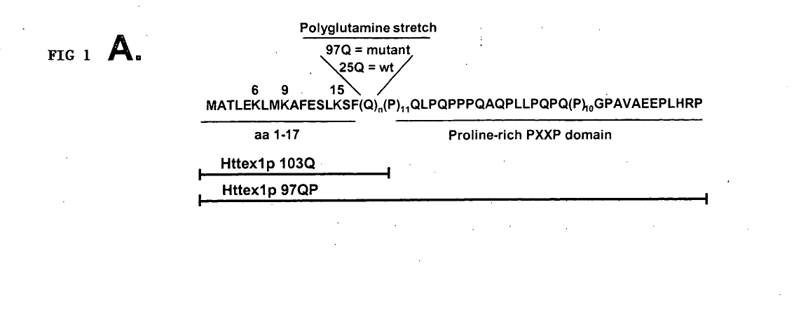

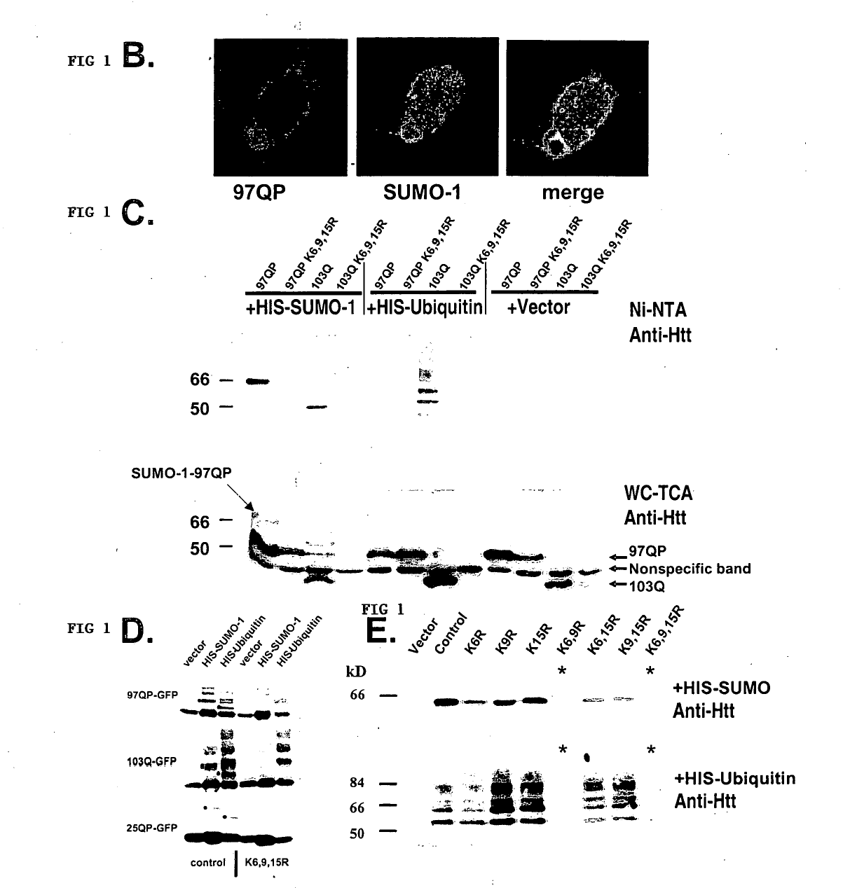

Immunofluorescence Analysis. For experiments demonstrating co-localization of Htt with SUMO-1, immortalized striatal cells were permeabilized in 0.05% digitonin (Sigma) in PBS on ice for 5 min and washed in ice-cold PBS before fixation with 4% paraformaldehyde for 10 min and methanol for 2 min. The coverslips were incubated with the primary antibody followed by the Texas red (TR) and fluorescein (FITC) conjugated secondary antibodies. FITC conjugated anti-sheep (1:5000), FITC conjugated anti-rabbit (1:5000) and TR conjugated anti-mouse (1:1000) was from Jackson ImmunoResearch Laboratories. Next, the cells were incubated with DAPI and mounted in Vectashield (Vector Laboratories Inc). The cells were analyzed with Zeiss Axiovert 25 inverted microscope using AxioVision 3.0 imaging system (Carl Zeiss). Immunofluorescence (FIG. 1B) to demonstrate co-localization of Httex1p and SUMO-1 was done using mouse anti-Htt EM48 antibody (Chemicon MAB 5374) and rabbit anti-SUMO-1 antibody (Santa Cruz Biotechnology FL-101). Immunofluorescence analysis in FIG. 2D was done with S830 sheep anti-Htt antibody (1:500) and FITC secondary antibody. Immunofluorescence analysis in FIG. 2E was done with anti-Htt CAG53 b antibody (1:5000) and Texas Red secondary antibody.

Unexpanded Httex1p is SUMOylated. The first exon of Htt encodes only 3 lysine residues: K6, K9, and K15 (FIG. 1A). None of these lysines are within a precise ΨKXE consensus SUMOylation target sequence, Ψ being a hydrophobic residue and X any residue (A. Verger, J. Perdomo, M. Crossley, EMBO Rep 4, 137-42 (2003)), however there is precedence for non-consensus SUMOylation sites [e.g. K65 of PML (T. Kamitani et al., J Biol Chem 273, 26675-82 (1998)) and PCNA (C. Hoege, B. Pfander, G. L. Moldovan, G. Pyrowolakis, S. Jentsch, Nature 419, 135-41 (2002))]. To determine if mutant Htt, previously shown to be ubiquitinated, can also be SUMOylated, we expressed HIS-SUMO-1 or HIS-ubiquitin in HeLa cells that were co-transfected with plasmids expressing expanded Httex1p either unmodified or with the 3 lysines mutated to arginine (K6,9,15R). We used magnetic nickel columns under denaturing conditions (Ni-NTA, Qiagen) to purify proteins covalently linked to HIS-SUMO-1 or HIS-ubiquitin followed by Western analysis with anti-Htt antibody to test for SUMOylation and ubiquitination of Httex1p. To eliminate any potential complications from SUMOylation of protein tags, no epitope tag was used in these experiments. We found 97QP and 103Q could both be SUMOylated and ubiquitinated, dependent on the presence of lysines 6, 9, and 15 (FIG. 1C and 1E). However, since the sensitivity of detection is greater when proteins are epitope-tagged, the experiments were also performed using polyQ Httex1p-GFP fusion proteins. Consistent with above results, higher molecular weight proteins corresponding to SUMOylated or ubiquitinated forms of both expanded and unexpanded polyglutamine versions of these fusions are observed in the presence of exogenous HIS-SUMO-1 or HIS-ubiquitin (FIG. 1D). This experiment demonstrates that unexpanded polyQ-repeat Httex1p (25QP-GFP) can be SUMO-1 modified as well as expanded polyQ 97QP-GFP and 103Q-GFP; we were unable to show this with untagged 25QP due to its low abundance in cellular extracts.

Huntingtin can be modified by SUMO-1 or Ubiquitin and co-localizes with SUMO-1 in cell culture. Truncated Htt (Httex1p 97QP, FIG. 1A) and HIS-SUMO-1 co-localize when transfected into immortalized striatal nerve cells, 12.7 (FIG. 1B), reflecting either direct modification of Htt by SUMO-1 or colocalization of Htt with other SUMOylated proteins. To identify possible modifications of mutant Httex1p which contains only 3 lysine residues, K6, K9, and K15 (FIG. 1A), HIS-SUMO-1 or HIS-ubiquitin was co-expressed with Httex1p either intact or with the lysines mutated to arginine (K6,9,15R) in both HeLa and striatial cells. We also compared Htt fragments either with or without the proline-rich domain immediately following the Qs, e.g. 97QP and 103Q respectively (FIG. 1A). Both proteins can be-SUMOylated or ubiquitinated (FIG. 1C, lower panel) and a single primary SUMOylated species predominates, although more complex SUMO or ubiquitin modifications can be seen (FIG. 1D). The low levels of Htt detected in the HIS-ubiquitin enriched fraction of cells transfected with the K6,9,15R triple mutant (FIG. 1E) may reflect ubiquitination of the amino-terminus of the protein. The proline motif enhances SUMO-1 modification but suppresses ubiquitination, consistent with a possible influence of other proteins that interact with this region.

Huntingtin is stabilized by SUMO-1 modification and fusion of SUMO-1 to Httex1p or deletion of the proline-rich region of Httex1p decreases inclusion formation. All possible combinations of lysine mutants were generated within the 1 st 17 amino acids of 97QP Httex1p, revealing that common residues are targeted for both SUMOylation and ubiquitination and specifically implicating residues K6 and K9 in these processes. When the lysine residues of Htt are mutated (double and triple lysine mutants) the abundance of Httex1p protein is reduced (FIG. 2A). Further, the proline-rich region of Httex1p also appears to contribute to the observed increase in soluble protein level since Htt peptides with the prolines are more abundant than those without.

We explored 3 possible mechanisms whereby SUMO modification might influence pathology, namely aggregation, subcellular localization and transcriptional dysregulation. To isolate the effects of SUMO modification, we fused SUMO to the amino-terminus of Htt (S. Ross, et al., (2002) Mol Cell 10, 831-42) and compared accumulation with and without the lysines and/or SUMO-1 fusion (FIG. 2B). The Htt peptide accumulates dramatically when fused to SUMO while the elimination of SUMOylation sites decreases levels.

Fusion of SUMO-1 to Httex1p also affects the aggregation properties of the protein. Using a dividing striatal neuronal cell line where nuclear localization is minimal, expression of Httex1p 97QP leads to the formation of large, Htt aggregates or inclusions (FIG. 2D; 97QP and 97QP K6,9,15R). Aggregate formation is also evident as Htt positive material that fails to penetrate the polyacrylamide gels of Western blots (FIG. 2B 97QP and 97QP K6,9,15R). However, when “permanently” SUMOylated, the levels of disperse cytoplasmic protein are increased and inclusions are reduced (FIG. 2D compare 97QP to SUMO-97QP, and 97QP K6,9,15R to SUMO-97QP K6,9,15R). In addition, aggregates of both SUMO-97QP and SUMO-97QP K6,9,15R are absent from the stacking gels of Western blots and soluble levels are increased (FIG. 2B). Thus, surprisingly, in addition to stabilizing the protein, SUMOylation appears to reduce the formation of visible, SDS-insoluble aggregates and increase disperse Htt staining in cells.

When the proline-rich domain is absent, (103Q) expression of Httex1p does not produce visible inclusions or SDS insoluble material (FIG. 2B,E), although when 103Q is fused to GFP numerous inclusions are evident, indicating that when not fused to GFP, inclusion formation is dependent on the presence of the proline-rich region (FIG. 2E). We considered whether the apparent SUMO-induced increase in Htt stability might simply reflect a redistribution of Htt from inclusions to soluble material. However, because levels of 103Q also increase when permanently SUMOylated, even though Q103 does not form inclusions (FIG. 2B, E), this is not the case. We conclude that the proline-rich region of Httex1p is essential for inclusion formation and that SUMOylation of Httex1p both stabilizes the protein and reduces visible inclusion formation. It has been reported that ubiquitination of polyQ proteins can trigger formation of visible protein aggregates. If ubiquitination is essential for inclusion formation, then blocking the putative ubiquitination sites by SUMOylation might alter the protein aggregation state as observed. In any case, the addition of SUMO appears to cause Htt to accumulate in a non-aggregated or early aggregation state that is not visible by light microscopy. In light of recent studies showing that soluble oligomers that precede aggregates may be the toxic species, SUMO modification of Htt could be increasing the levels of these potentially toxic oligomers.

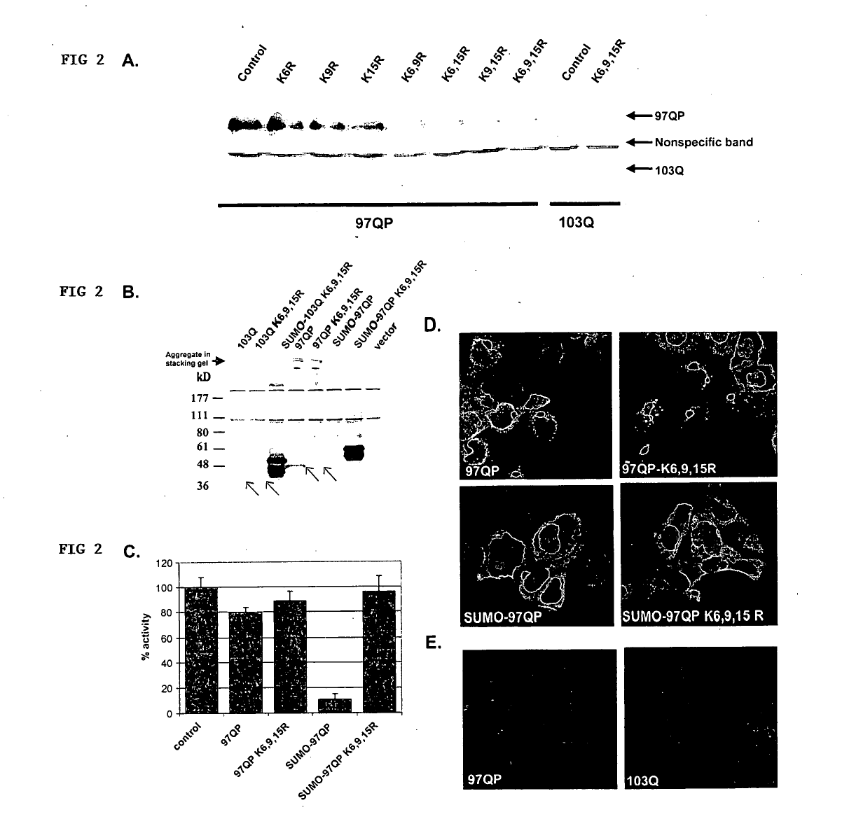

Permanently SUMOylated Httex1p dramatically represses transcription. Pathogenic processes in HD and other polyQ disorders appear to include repression of transcription by the mutant protein. Since SUMO-1 has been shown to play a role in transcriptional regulation through modification of transcription factors, we asked whether SUMOylation of Htt increases transcriptional repression. Because the fraction of Htt that is SUMOylated is quite low, we used permanently SUMOylated Htt to test the effects of modified Htt on transcriptional activity. We have previously shown that the WAF1-pGL3 and the Multi Drug Resistance 1 (MDR1) gene promoters are repressed by expanded Htt (J. S. Steffan et al., (2000) Proc Natl Acad Sci U S A 97, 6763-68). When transfected into striatal cells, SUMO-97QP appears to dramatically repress both the MDR1 and WAF1-pGL3 promoters that are only modestly repressed by Httex1p 97QP (FIG. 3). These results are very similar to those in FIG. 2C, demonstrating dramatic repression of the CMV promoter by SUMO-97QP. These observations demonstrate that SUMO modification of Htt can substantially increase the suppressive effect of Htt on transcription, even with minimal SUMO-97QP protein levels present.

How might SUMOylation affect transcription? SUMOylation could cause increased nuclear or subnuclear localization of SUMOylated Httex1p, since SUMOylated proteins frequently localize to PML nuclear bodies that are implicated in transcriptional regulation. Modification of Htt by SUMO may increase the ability of Htt to be recruited to transcriptional repression complexes on chromatin. Alternatively, SUMOylation might alter cytoplasmic activity of Htt and either cause the release of a nuclear repressor that translocates to the nucleus or cause the cytoplasmic retention of proteins necessary to activate transcription, e.g. CBP.

The CMV promoter is repressed by SUMO-97QP. Control experiments to detect potential effects of these constructs on the expression of the CMV promoter driving the Htt transgene demonstrate that 97QP and 97QP K6,9,15R only modestly repress and rescue CMV expression respectively (FIG. 2C). Such minor effects would be expected if only a small fraction of Htt is SUMOylated as indicated in FIG. 1C. In contrast, SUMOylated 97QP (SUMO-97QP) significantly represses expression from the CMV promoter. Notably, repression is largely abolished by mutation of the lysines even when SUMO is fused to the protein indicating that SUMO modification is necessary but not alone sufficient to affect repression. This observation suggests that two structural elements are required for transcriptional repression, namely SUMO modification and a structurally intact 1-17 amino acid domain. The repression of CMV by SUMO-97QP means that any potential accumulation of the SUMO-97QP protein is masked by the self-repression of the transgene (FIG. 2C). The steady state levels of SUMO-modified Htt (in the absence of direct fusion) are quite low, presumably due to competition with ubiquitination, limited SUMOylation and significant isopeptidase-mediated removal of SUMO; thus the permanent attachment of SUMO to all of the Htt protein reveals the dramatic stabilization that this modification can exert even when not in its normal side chain position(s). Chronic low level SUMOylation as seen in normal cells might be expected to contribute to progressive pathology of HD.

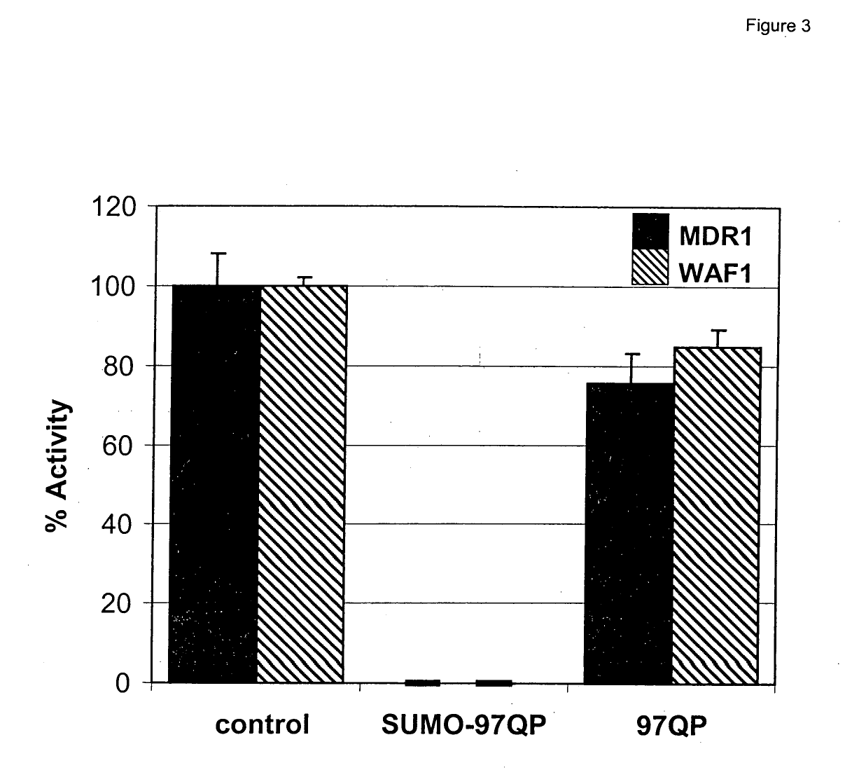

Amino acids 1-17 of Htt comprise a cytoplasmic targeting sequence. Since SUMO modification occurs in the first 17 amino acids of Htt, we asked whether this region influences subcellular localization of Htt since SUMO-1 modification can influence nuclear localization of proteins. We find that the first 17 amino acids of Htt can target proteins to the cytosol even when challenged with a strong Nuclear Localization Sequence (NLS). Specifically, when GFP is fused to the SV40NLS, its nuclear localization is greatly enhanced (FIG. 4). However, the addition of the 1 st 17 amino acids of Htt to the amino-terminus efficiently relocalizes this protein to the cytoplasm. Further, Httex1p, even when fused to an NLS, can be targeted to the cytosol when the first 17 amino acids of Htt are present compared to complete nuclear localization when they are absent.

To further define the mechanism by which amino acids 1-17 of Htt accomplish cytosolic targeting, we assessed whether this sequence could act as an export receptor (CRM-1) dependent nuclear export signal (NES). Several large hydrophobic amino acids characteristic of an NES are indeed found in the 1 st 17 amino acids of Htt. CRM-1 mediated export is abolished by Leptomycin B (LMB) treatment, therefore the effect of LMB on nuclear exclusion mediated by Htt 1-17 was tested. As expected, the nuclear exclusion of a control NES-GFP was efficiently reversed by LMB addition (FIG. 4) whereas the localization of GFP or NLS-GFP was unaffected. Surprisingly, LMB had no effect upon the nuclear exclusion of Htt 1-17-NLS-GFP, suggesting that Htt 1-17 either mediates a novel CRM-1 independent export function or it confers a cytoplasmic retention signal.

Interestingly, the two-amino acid spacing of the last two hydrophobic amino acids of Htt 1-17 (FX2L) are indeed in disagreement with the NES consensus sequence (LX(1-3)LX(2-3)LXL, L being a large hydrophobic residue). A possible mechanism whereby SUMO modification can enhance pathology is by masking of this cytoplasmic retention sequence. The demonstration that these 17 amino acids of Htt comprise a cytosolic retention sequence and also contain residues that can be SUMOylated raises the possibility that the SUMO modification described here may alter subcellular accumulation of Htt as well as modulate other properties of expanded Htt.

The first 17 amino acids of Htt can target proteins to the cytosol even when challenged with a strong Nuclear Localization Sequence (NLS). Since SUMO-1 modification can influence nuclear localization of proteins and SUMO modification occurs on the first 17 amino acids of Htt, we asked whether this region influences subcellular localization. We find that the first 17 amino acids of Htt can target proteins to the cytosol even when challenged with a strong Nuclear Localization Sequence (NLS). This targeting may involve a novel CRM-1 independent export function or it may involve a cytoplasmic retention signal (FIG. 4). In human patient brain tissue, mouse models and cell culture, mutant Htt protein is progressively localized from the cytoplasm to the nucleus. If nuclear localization of Htt is essential for HD pathogenesis, it is paradoxical that the pathogenic Htt fragment contains a cytoplasmic targeting signal. On the other hand, SUMO modification might preferentially mask this cytoplasmic retention signal in some cell types more than in others, allowing for different levels of nuclear localization and selective neuronal toxicity.

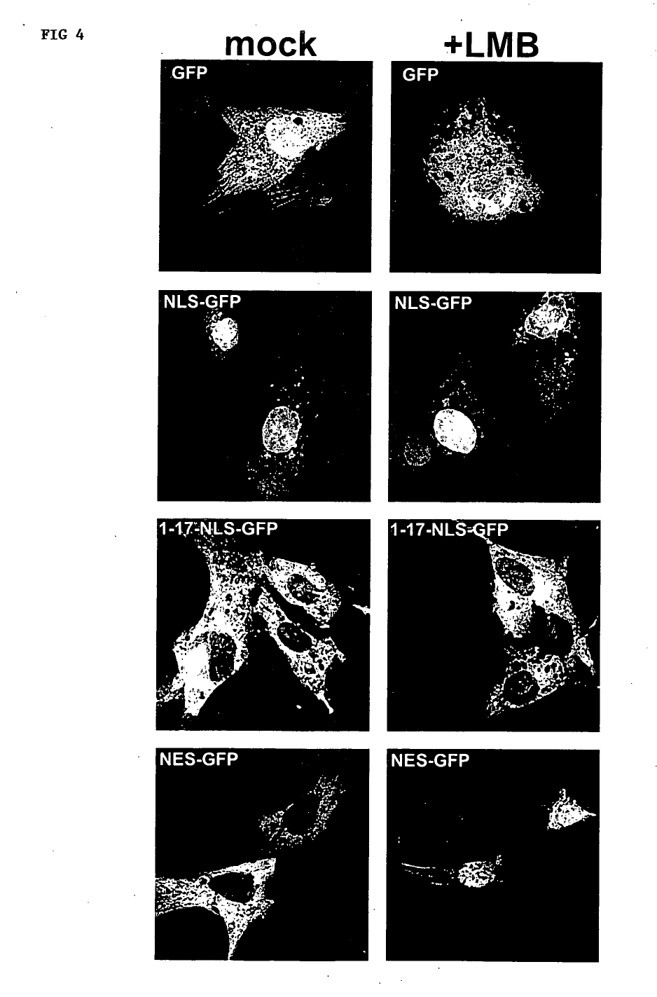

Genetic reduction of SUMO activity in Drosophila reduces neurodegeneration in an HD fly model. We next sought to genetically determine the relative contributions of SUMO and ubiquitin to pathogenesis. When mutant Httex1p (93QP) is expressed in all neurons of Drosophila, photoreceptor neurons are progressively lost and the integrity of the eye is compromised. However, when such animals are compared to sibs with reduced SUMOylation activity (i.e. heterozygous for a SUMO mutant, smt3/+), neurodegeneration is significantly reduced (FIG. 5A). In similar experiments, the level of ubiquitination activity was reduced and found to make pathology modestly worse (FIG. 5B). Thus SUMOylation makes pathology significantly worse while ubiquitination makes pathology modestly better.

Since the same lysines are targeted by both SUMOylation and ubiquitination, and since the global genetic reduction of both SUMO and ubiquitin can impact many cellular proteins and thereby indirectly affect the pathology of Htt, we sought to directly determine the influence of Htt modification on toxicity. Transgenic flies expressing Httex1p 97QP with or without the lysines mutated were compared. Mutation of the lysine residues dramatically reduces pathology (FIG. 5C). Similar experiments using the gmr-GAL4 driver that expresses the transgenes in all cells of the Drosophila compound eye confirmed that mutation of the lysine residues dramatically suppresses cytotoxicity (FIG. 5D), indicating that the availability of these lysines is essential to the pathogenic process.

Is the role of SUMOylation simply to prevent ubiquitination? If mutating the lysines served only to reduce ubiquitination, then pathology should worsen. Instead, the exact opposite is true, namely, pathology is dramatically reduced when the lysines are no longer available for post-translational modification. These data indicate that the inability to be SUMOylated has a more dramatic impact on pathology than the reduced ability to be ubiquitinated and degraded.

These observations demonstrate that Htt can be SUMOylated and suggest that SUMOylation can increase Htt accumulation, decrease aggregate formation and possibly increase toxic oligomers, potentially mask a cytoplasmic retention signal and increase nuclear repression of transcription. The impact of SUMOylation on HD pathogenesis in vivo is dramatic.

Decreasing expression of the SUMO-1 precursor, inhibiting SUMO-1 ligases, or increasing isopeptidase activity to remove SUMO-1 could each reduce the level of SUMOylated Htt in the cell and suppress pathogenesis. The E3 ligase specific for attachment of SUMO-1 to Htt presents a particularly attractive therapeutic target.

Drug Screening Assays. Transgenic animals, such as the Drosophila HD model described herein and in the references cited herein, may be used to identify compounds that reduce SUMOylation of mutant polyglutamine repeat proteins and which, therefore, are potentially useful in the treatment of HD and other polyQ-associated diseases. For example, fly HD models may be treated with various candidate compounds and the resulting effect, if any, on the rescue of neurodegeneration in the fly eye evaluated. The effect of candidate compounds on SUMOylation of Huntingtin and other polyglutamine repeat proteins may also be measured directly, using techniques known in the art. Preferably, the compounds screened are suitable for use in humans.

Drug screening assays in general suitable for use with transgenic animals are known. See, for example, U.S. Pat. Nos. 6,028,245 and 6,455,757. Various methods suitable for screening the efficacy of candidate compounds in reducing SUMOylation of glutamine repeat proteins are described herein or in the references cited herein. However, it will be understood by one of skill in the art that many other assays may also be used. Candidate compounds may be screened for their direct effect of the SUMOylation of polyglutamine repeat proteins or the screen can employ any phenomena associated with HD pathology that can be readily assessed in an animal model.

Therapeutic Agents. Once compounds have been identified in drug screening assays as eliminating or ameliorating the effects of HD pathologies, these compounds can be used as therapeutic agents, provided they are biocompatible with the animals, preferably humans, to whom they are administered.

The therapeutic agents of the present invention can be formulated into pharmaceutical compositions by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. Administration of the compounds can be administered in a variety of ways known in the art, as, for example, by oral, buccal, rectal, parenteral, intraperitoneal, intradermal, transdermal, intratracheal, etc., administration.

Depending upon the particular route of administration, a variety of pharmaceutically acceptable carriers, well known in the art can be used. These carriers include, but are not limited to, sugars, starches, cellulose and its derivatives, malt, gelatin, talc, calcium sulfate, vegetable oils, synthetic oils, polyols, alginic acid, phosphate buffered solutions, emulsifiers, isotonic saline, and pyrogen-free water. Preservatives and other additives can also be present. For example, antimicrobial, antioxidant, chelating agents, and inert gases can be added (see, generally, Remington's Pharmaceutical Sciences, 16th Edition, Mack, (1980)).

The concentration of therapeutically active compound in the formulation may vary from about 0.1-100 wt. %.

Those of skill will readily appreciate that dose levels can vary as a function of the specific therapeutic agents, the severity of the symptoms and the susceptibility of the subject to side effects. Preferred dosages for a given therapeutic agent are readily determinable by those of skill in the art by a variety of means. A preferred means is to measure the physiological potency of a given therapeutic agent.

The present inventors have shown that the Huntingtin protein can be SUMO-1 modified, that this modification stabilizes the protein, alters its aggregation and transcriptional repression properties and affects its pathogenic potential in a Drosophila model of HD.

In Huntington's disease (HD), pathogenic proteins with polyglutamine expansions accumulate. The present inventors have demonstrated that a pathogenic fragment of Huntingtin (Httex1p) can be modified either by SUMO-1 or by ubiquitin on identical lysines. In cultured cells, SUMOylation stabilizes Httex1p, affects transcriptional repression, and can alter the aggregation properties of Htt. In a Drosophila model of HD, SUMOylation exacerbates while ubiquitination relieves neurodegeneration. Further, mutations that prevent both SUMOylation and ubiquitination dramatically reduce pathology, indicating that the contribution of SUMOylation extends beyond simply preventing ubiquitination. Thus, treatments that modify SUMO activity should improve clinical outcomes in HD and other polyglutamine repeat diseases.

### References

Adamson, A. L. and Kenney, S. (2001). Epstein-barr virus immediate-early protein BZLF1 is SUMO-1 modified and disrupts promyelocytic leukemia bodies. J Virol 75, 2388-99.

Alefantis, T., Barmak, K., Harhaj, E. W., Grant, C. and Wigdahl, B. (2003). Characterization of a Nuclear Export Signal within the Human T Cell Leukemia Virus Type I Transactivator Protein Tax. J Biol Chem 278, 21814-22.

Apostol, B. L., Kazantsev, A., Raffioni, S., Illes, K., Pallos, J., Bodai, L., Slepko, N., Bear, J. E., Gertler, F. B., Hersch, S. et al. (2003). A cell-based assay for aggregation inhibitors as therapeutics of polyglutamine-repeat disease and validation in Drosophila. Proc Natl Acad Sci U S A 100, 5950-5.

Ariumi, Y., Kaida, A., Lin, J. Y., Hirota, M., Masui, O., Yamaoka, S., Taya, Y. and Shimotohno, K. (2000). HTLV-1 tax oncoprotein represses the p53-mediated trans-activation function through coactivator CBP sequestration. Oncogene 19, 1491-9.

Azimi, N., Nagai, M., Jacobson, S. and Waldmann, T. A. (2001). IL-15 plays a major role in the persistence of Tax-specific CD8 cells in HAM/TSP patients. Proc Natl Acad Sci U S A 98, 14559-64.

Bates, G. (2003). Huntingtin aggregation and toxicity in Huntington's disease. Lancet 361, 1642-4.

Bates, G., Harper, P. and Jones, L. (2002). Huntington's Disease. Oxford: Oxford University Press.

Bates, G. P., Mangiarini, L. and Davies, S. W. (1998). Transgenic mice in the study of polyglutamine repeat expansion diseases. Brain Pathol. 8, 699-714.

Bence, N. F., sampat, R. M. and Kopito, R. R. (2001). Impairment of the ubiquitin-proteasome system by protein aggregation. Science 292, 1552-1555.

Borden, K. L. (2002). Pondering the promyelocytic leukemia protein (PML) puzzle: possible functions for PML nuclear bodies. Mol Cell Biol 22, 5259-69.

Boutell, J. M., Thomas, P., Neal, J. W., Weston, V. J., Duce, J., Harper, P. S. and Jones, A. L. (1999). Aberrant interactions of transcriptional repressor proteins with the Huntington's disease gene product, huntingtin. Hum. Mol. Gen. 9, 1647-1655.

Carvalho, T., Seeler, J. S., Ohan, K., Jordan, P., Pettersson, U., Akusjarvi, G., Carmo-Fonseca, M. and Dejean, A. (1995). Targeting of adenovirus ElA and E4-ORF3 proteins to nuclear matrix-associated PML bodies. J Cell Biol 131, 45-56.

Cattaneo, E., Rigamonti, D., Goffredo, D., Zuccato, C., Squitieri, F. and Sipione, S. (2001). Loss of normal huntingtin function: new developments in Huntington's disease research. Trends Neurosci. 24, 1-8.

Cha, J.-H. (2000). Transcriptional dysregulation in Huntington's disease. Trends Neurosci. 2000, 387-392.

Chai, Y., Koppenhafer, S. L., Shoesmith, S. J., Perez, M. K. and Paulson, H. (1999). Evidence for proteasome involvement in polyglutamine disease: localization to nuclear inclusions in SCAa3/MJD and suppression of polyglutamine aggregation in vitro. Hum. Mol. Gen. 8, 673-682.

Chan, H. Y., Warrick, J. M., Andriola, I., Merry, D. and Bonini, N. M. (2002). Genetic modulation of polyglutamine toxicity by protein conjugation pathways in Drosophila. Hum Mol Genet 11, 2895-904.

Chan, H. Y. E., Warrick, J. M., Gray-Board, G. L., Paulson, H. and Bonini, N. M. (2000). Mechanisms of chaperone suppression of polyglutamine disease: selectivity, synergy, and modulation of protein solubility in Drosophila. Hum. Mol. Gen. 9, 2811-2820.

Chelbi-Alix, M. K. and de The, H. (1999). Herpes virus induced proteasome-dependent degradation of the nuclear bodies-associated PML and Sp100 proteins. Oncogene 18, 935-41.

Cummings, C. J., Mancini, M. A., Antalffy, B., DeFranco, D. B., Orr, H. T. and Zoghbi, H. Y. (1998). Chaperone suppression of aggregation and altered subcellular proteasome localization imply protein misfolding in SCA1. Nat. Gen. 19, 148-154.

Cummings, C. J., Reinstein, E., Sun, Y., Antalffy, B., Jiang, Y., Ciechanover, A., Orr, H. T., Beaudet, A. L. and Zoghbi, H. Y. (1999). Mutation of the E6-AP ubiquitin ligase reduces nuclear inclusion frequency while accelerating polyglutamine-induced pathology in SCA1 mice. Neuron 24, 879-92.

David, G., Neptune, M. A. and DePinho, R. A. (2002). SUMO-1 modification of histone deacetylase 1 (HDCA1) modulates its biological activities. J Biol Chem 277, 23658-63.

Davies, S. W., Turmaine, M., Cozens, B. A., Difiglia, M., Sharp, A. H., Ross, C. A., Scherzinger, E., Wanker, E. E., Mangiarini, L. and Bates, G. P. (1997). Formation of neuronal intranuclear inclusions underlies the neurological dysfunction in mice transgenic for the HD mutation. Cell 90, 537-548.

Desterro, J. M., Rodriguez, M. S. and Hay, R. T. (1998). SUMO-1 modification of IkappaBalpha inhibits NF-kappaB activation. Mol Cell 2, 233-9.

DiFiglia, M., Sapp, E., Chase, K. O., Davies, S. W., Bates, G. P., Vonsattel, J. P. and Aronin, N. (1997). Aggregation of huntingtin in neuronal intranuclear inclusions and dystrophic neurites in brain. Science 277, 1990-1993.

Donaldson, K. M., Li, W., Ching, K. A., Batalov, S., Tsai, C. C. and Joazeiro, C. A. (2003). Ubiquitin-mediated sequestration of normal cellular proteins into polyglutamine aggregates. Proc Natl Acad Sci U S A 100, 8892-8897.

Dunah, A. W., Jeong, H., Griffin, A., Kim, Y. M., Standaert, D. G., Hersch, S. M., Mouradian, M. M., Young, A. B., Tanese, N. and Krainc, D. (2002). Sp1 and TAFII130 transcriptional activity disrupted in early Huntington's Disease. Science 296, 2238-2243.

Ego, T., Ariumi, Y. and Shimotohno, K. (2002). The interaction of HTLV-1 Tax with HDAC1 negatively regulates the viral gene expression. Oncogene 21, 7241-6.

Ellis, M. C., O'Neill, E. M. and Rubin, G. M. (1993). Expression of Drosophila glass protein and evidence for negative regulation of its activity in non-neuronal cells by another DNA-binding protein. Development 119, 855-65.

Eskiw, C. H. and Bazett-Jones, D. P. (2002). The promyelocytic leukemia nuclear body: sites of activity? Biochem Cell Biol 80, 301-10.

Everett, R. D. (2001). DNA viruses and viral proteins that interact with PML nuclear bodies. Oncogene 20, 7266-73.

Everett, R. D., Freemont, P., Saitoh, H., Dasso, M., Orr, A., Kathoria, M. and Parkinson, J. (1998). The disruption of ND10 during herpes simplex virus infection correlates with the Vmw110- and proteasome-dependent loss of several PML isoforms. J Virol 72, 6581-91.

Fabunmi, R. P., Wigley, W. C., Thomas, P. J. and DeMartino, G. N. (2001). Interferon gamma regulates accumulation of the proteasome activator PA28 and immunoproteasomes at nuclear PML bodies. J Cell Sci 114, 29-36.

Ferbeyre, G. (2002). PML a target of translocations in APL is a regulator of cellular senescence. Leukemia 16, 1918-26.

Fernandez-Funez, P., Nino-Rosales, M. L., Gouyon, B., She, W.-C., Luchak, J. M., Martinez, P., Turleganos, E., Benito, J., Capovilla, M., Skinner, P. J. et al. (2000). Identification of genes that modify ataxin-1-induced neurodegeneration. Nature 408, 101-106.

Ferrante, R. J., Kubilus, J. K., Lee, J., Ryu, H., Beesen, A., Zucker, B., Smith, K., Kowall, N. W., Ratan, R. R., Luthi-Carter, R. et al. (2003). Histone deacetylase inhibition by sodium butyrate chemotherapy ameliorates the neurodegenerative phenotype in Huntington's disease mice. J Neurosci 23, 9418-27.

Fuchsova, B., Novak, P., Kafkova, J. and Hozak, P. (2002). Nuclear DNA helicase II is recruited to IFN-alpha-activated transcription sites at PML nuclear bodies. J Cell Biol 158, 463-73.

Gallimore, P. H. and Turnell, A. S. (2001). Adenovirus E1A: remodelling the host cell, a life or death experience. Oncogene 20, 7824-35.

Girdwood, D., Bumpass, D., Vaughan, O. A., Thain, A., Anderson, L. A., Snowden, A. W., Garcia-Wilson, E., Perkins, N. D. and Hay, R. T. (2003). P300 transcriptional repression is mediated by SUMO modification. Mol Cell 11, 1043-54.

Glickman, M. H. and Ciechanover, A. (2002). The ubiquitin-proteasome proteolytic pathway: destruction for the sake of construction. Physiol Rev 82, 373-428.

Gunawardena, S., Her, L. S., Brusch, R. G., Laymon, R. A., Niesman, I. R., Gordesky-Gold, B., Sintasath, L., Bonini, N. M. and Goldstein, L. S. (2003). Disruption of axonal transport by loss of huntingtin or expression of pathogenic polyQ proteins in Drosophila. Neuron 40, 25-40.

Harjes, P. and Wanker, E. E. (2003). The hunt for huntingtin function: interaction partners tell many different stories. Trends Biochem Sci 28, 425-33.

Harrod, R., Kuo, Y. L., Tang, Y., Yao, Y., Vassilev, A., Nakatani, Y. and Giam, C. Z. (2000). p300 and p300/cAMP-responsive element-binding protein associated factor interact with human T-cell lymphotropic virus type-1 Tax in a multi-histone acetyltransferase/activator-enhancer complex. J Biol Chem 275, 11852-7.

Harrod, R., Nacsa, J., Van Lint, C., Hansen, J., Karpova, T., McNally, J. and Franchini, G. (2002). Human immunodeficiency virus type-1 Tat/Co-activator acetyltransferase interactions inhibit p53K320-acetylation and p53-responsive transcription. J Biol Chem.

Hockly, E., Richon, V. M., Woodman, B., Smith, D. L., Zhou, X., Rosa, E., Sathasivam, K., Ghazi-Noori, S., Mahal, A., Lowden, P. A. et al. (2003). Suberoylanilide hydroxamic acid, a histone deacetylase inhibitor, ameliorates motor deficits in a mouse model of Huntington's disease. Proc Natl Acad Sci U S A 100, 2041-6.

Hoege, C., Pfander, B., Moldovan, G. L., Pyrowolakis, G. and Jentsch, S. (2002). RAD6-dependent DNA repair is linked to modification of PCNA by ubiquitin and SUMO. Nature 419, 135-41.

Holbert, S., Denghien, I., Kiechle, T., Rosenblatt, A., Wellington, C., Hayden, M. R., Margolis, R. L., Ross, C. A., Dausset, J., Ferrante, R. J. et al. (2001). The Gln-Ala repeat transcriptional activator CA150 interacts with huntingtin: neuropathologic and genetic evidence for a role in Huntington's disease pathogenesis. Proc Natl Acad Sci U S A 98, 1811-6.

Hughes, R. E. (2002). Polyglutamine disease: acetyltransferases awry. Curr Biol 12, R141-3.

Hughes, R. E., Lo, R. S., Davis, C., Strand, A. D., Neal, C. L., Olson, J. M. and Fields, S. (2001). Altered transcription in yeast expressing expanded polyglutamine. Proc Natl Acad Sci U S A 98, 13201-6.

Hughes, R. E. and Olson, J. M. (2001). Therapeutic opportunities in polyglutamine disease. Nat Med 7, 419-23.

Ishov, A. M., Sotnikov, A. G., Negorev, D., Vladimirova, O. V., Neff, N., Kamitani, T., Yeh, E. T., Strauss, J. F., 3rd and Maul, G. G. (1999). PML is critical for ND10 formation and recruits the PML-interacting protein daxx to this nuclear structure when modified by SUMO-1. J Cell Biol 147, 221-34.

Kahyo, T., Nishida, T. and Yasuda, H. (2001). Involvement of PIAS1 in the sumoylation of tumor suppressor p53. Mol Cell 8, 713-8.

Kalchman, M. A., Graham, R. K., Xia, G., Koide, H. B., Hodgson, J. G., Graham, K. C., Goldberg, Y. P., Gietz, R. D., Pickart, C. M. and Hayden, M. R. (1996). Huntingtin is ubiquitinated and interacts with a specific ubiquitin-conjugating enzyme. J Biol Chem 271, 19385-94.

Kamitani, T., Kito, K., Nguyen, H. P., Wada, H., Fukuda-Kamitani, T. and Yeh, E. T. (1998). Identification of three major sentrinization sites in PML. J Biol Chem 273, 26675-82.

Kao, S. Y., Lemoine, F. J. and Marriott, S. J. (2001). p53-independent induction of apoptosis by the HTLV-I tax protein following UV irradiation. Virology 291, 292-8.

Kayed, R., Head, E., Thompson, J. L., McIntire, T. M., Milton, S. C., Cotman, C. W. and Glabe, C. G. (2003). Common structure of soluble amyloid oligomers implies common mechanism of pathogenesis. Science 300, 486-9.

Kaytor, M. D., Duvick, L. A., Skinner, P. J., Koob, M. D., Ranum, L. P. and Orr, H. T. (1999). Nuclear localization of the spinocerebellar ataxia type 7 protein, ataxin-7. Hum Mol Genet 8, 1657-64.

Kazantsev, A., Preisinger, E., Dranovsky, A., Goldgaber, D. and Housman, D. (1999). Insoluble detergent-resistant aggregates form between pathological and nonpathological lengths of polyglutamine in mammalian cells. Proc. Natl. Acad. Sci. USA 96, 11404-11409.

Kazantsev, A., Walker, H. A., Slepko, N., Bear, J. E., Preisinger, E., Steffan, J. S., Zhu, Y. Z., Gertler, F. B., Housman, D. E., Marsh, J. L. et al. (2002). A bivalent Huntingtin binding peptide suppresses polyglutamine aggregation and pathogenesis in Drosophila. Nat Genet 30, 367-376.

Kegel, K. B., Meloni, A. R., Yi, Y., Kim, Y. J., Doyle, E., Cuiffo, B. G., Sapp, E., Wang, Y., Qin, Z. H., Chen, J. D. et al. (2002). Huntingtin is present in the nucleus, interacts with the transcriptional corepressor C-terminal binding protein, and represses transcription. J Biol Chem 277, 7466-76.

Khoshnan, A., Ko, J. and Patterson, P. H. (2002). Effects of intracellular expression of anti-huntingtin antibodies of various specificities on mutant huntingtin aggregation and toxicity. Proc Natl Acad Sci U S A 99, 1002-7.

Kirsh, O., Seeler, J. S., Pichler, A., Gast, A., Muller, S., Miska, E., Mathieu, M., Harel-Bellan, A., Kouzarides, T., Melchior, F. et al. (2002). The SUMO E3 ligase RanBP2 promotes modification of the HDAC4 deacetylase. Embo J 21, 2682-91.

Klement, I. A., Skinner, P. J., Kayton, M. D., Hong, Y., Hersch, S. M., Clark, H. B., Zoghbi, H. Y. and Orr, H. T. (1998). Ataxin-1 nuclear localization and aggregation: role in polyglutamine-induced disease in SCA1 transgenic mice. Cell 94, 41-53.

Lallemand-Breitenbach, V., Zhu, J., Puvion, F., Koken, M., Honore, N., Doubeikovsky, A., Duprez, E., Pandolfi, P. P., Puvion, E., Freemont, P. et al. (2001). Role of promyelocytic leukemia (PML) sumolation in nuclear body formation, 11S proteasome recruitment, and As203-induced PML or PML/retinoic acid receptor alpha degradation. J Exp Med 193, 1361-71.

Langley, E., Pearson, M., Faretta, M., Bauer, U. M., Frye, R. A., Minucci, S., Pelicci, P. G. and Kouzarides, T. (2002). Human SIR2 deacetylates p53 and antagonizes PML/p53-induced cellular senescence. Embo J 21, 2383-96.

Li, H., Leo, C., Zhu, J., Wu, X., O'Neil, J., Park, E. J. and Chen, J. D. (2000). Sequestration and inhibition of Daxx-mediated transcriptional repression by PML. Mol Cell Biol 20, 1784-96.

Li, S.-H., Cheng, A. L., Zhou, H., Lam, S., Rao, M., Li, H. and Li, X.-J. (2002). Interaction of Huntington disease protein with transcriptional activator Sp1. Mol. Cell. Biol. 22, 1277-1287.

Li, Y., Wang, H., Wang, S., Quon, D., Liu, Y. W. and Cordell, B. (2003). Correction for the article “Positive and negative reglation of APP amyloidogenesis by sumoylation. Proc Natl Acad Sci U S A 100, 9102.

Li, Y., Wang, H., Wang, S., Quon, D., Liu, Y. W. and Cordell, B. (2003). Positive and negative regulation of APP amyloidogenesis by sumoylation. Proc Natl Acad Sci U S A 100, 259-64.

Lin, R. J., Sternsdorf, T., Tini, M. and Evans, R. M. (2001). Transcriptional regulation in acute promyelocytic leukemia. Oncogene 20, 7204-15.

Lin, X., Liang, M., Liang, Y. Y., Brunicardi, F. C. and Feng, X. H. (2003). SUMO-1/Ubc9 promotes nuclear accumulation and metabolic stability of tumor suppressor Smad4/DPC4. J Biol Chem 278, 31043-31048.

Luo, L., Liao, Y. J., Jan, L. Y. and Jan, Y. N. (1994). Distinct morphogenetic functions of similar small GTPases: Drosophila Drac1 is involved in axonal outgrowth and myoblast fusion. Genes Dev 8, 1787-802.

Luthi-Carter, R. and Cha, J.-H. (2003). Mechanisms of transcriptional dysregulation in Huntington's disease. Neursci Res 3, 165-177.

Luthi-Carter, R., Strand, A., Peters, N. L., Solano, S. M., Hollingsworth, Z. R., Menon, A. S., Frey, A. S., Spektor, B. S., Penney, E. B., Schilling, G. et al. (2000). Decreased expression of striatal signaling genes in a mouse model of Huntington's disease. Hum. Mol. Gen. 9, 1259-1267.

Mangiarini, L., Sathasivam, K., Seller, M., Cozens, B., Harper, A., Hetherington, C., Lawton, M., Trottier, Y., Lehrach, H., Davies, S. W. et al. (1996). Exon 1 of the HD gene with an expanded CAG repeat is sufficient to cause a progressive neurological phenotype in transgenic mice. Cell 87, 493-506.

Margolis, R. L. and Ross, C. A. (2001). Expansion explosion: new clues to the pathogenesis of repeat expansion neurodegenerative diseases. Trends Mol Med 7, 479-82.

Marsh, J. L., Walker, H., Theisen, H., Zhu, Y.-Z., Fielder, T., Purcell, J. and Thompson, L. M. (2000). Expanded polyglutamine peptides alone are intrinsically cytotoxic and cause neurodegeneration in Drosophila. Hum. Mol. Gen. 9, 13-25.

McCampbell, A. and Fischbeck, K. H. (2001). Polyglutamine and CBP: fatal attraction? Nat Med 7, 528-30.

McCampbell, A., Taye, A. A., Whitty, L., Penney, E., Steffan, J. S. and Fischbeck, K. H. (2001). Histone deacetylase inhibitors reduce polyglutamine toxicity. Proc Natl Acad Sci U S A 98, 15179-84.

McCampbell, A., Taylor, J. P., Taye, A. A., Robitschek, J., Li, M., Walcott, J., Merry, D., Chai, Y., Paulson, H., Sobue, G. et al. (2000). CREB-binding protein sequestration by expanded polyglutamine. Hum Mol Genet 9, 2197-202.

Melchior, F. (2000). SUMO-nonclassical ubiquitin. Annu Rev Cell Dev Biol 16, 591-626.

Miller, J. H. (1972). Unit VII: Assays of the lac operon enzymes, experiment 48: Assay of beta-galactosidase. Experiments in molecular-genetics, 352-355.

Mitsui, K., Nakayama, H., Akagi, T., Nekooki, M., Ohtawa, K., Takio, K., Hashikawa, T. and Nukina, N. (2002). Purification of polyglutamine aggregates and identification of elongation factor-1alpha and heat shock protein 84 as aggregate-interacting proteins. J Neurosci 22, 9267-77.

Moulignier, A., Moulonguet, A., Pialoux, G. and Rozenbaum, W. (2001). Reversible ALS-like disorder in HIV infection. Neurology 57, 995-1001.

Muller, S., Berger, M., Lehembre, F., Seeler, J. S., Haupt, Y. and Dejean, A. (2000). c-Jun and p53 activity is modulated by SUMO-1 modification. J Biol Chem 275, 13321-9.

Muller, S. and Dejean, A. (1999). Viral immediate-early proteins abrogate the modification by SUMO-1 of PML and Sp100 proteins, correlating with nuclear body disruption. J Virol 73, 5137-43.

Muller, S., Hoege, C., Pyrowolakis, G. and Jentsch, S. (2001). SUMO, ubiquitin's mysterious cousin. Nat Rev Mol Cell Biol 2, 202-10.

Muller, S., Matunis, M. J. and Dejean, A. (1998). Conjugation with the ubiquitin-related modifier SUMO-1 regulates the partitioning of PML within the nucleus. Embo J 17, 61-70.

Muslin, A. J. and Xing, H. (2000). 14-3-3 proteins: regulation of subcellular localization by molecular interference. Cell Signal 12, 703-9.

Negorev, D. and Maul, G. G. (2001). Cellular proteins localized at and interacting within ND10/PML nuclear bodies/PODs suggest functions of a nuclear depot. Oncogene 20, 7234-42.

Nicot, C. and Harrod, R. (2000). Distinct p300-responsive mechanisms promote caspase-dependent apoptosis by human T-cell lymphotropic virus type 1 Tax protein. Mol Cell Biol 20, 8580-9.

Nucifora, F. C., Jr., Sasaki, M., Peters, M. F., Huang, H., Cooper, J. K., Yamada, M., Takahashi, H., Tsuji, S., Troncoso, J., Dawson, V. L. et al. (2001). Interference by huntingtin and atrophin-1 with cbp-mediated transcription leading to cellular toxicity. Science 291, 2423-8.

Pandolfi, P. P. (2001). Histone deacetylases and transcriptional therapy with their inhibitors. Cancer Chemother Pharmacol 48 Suppl, S17-9.

Paulson, H. L., Das, S. S., Crino, P. B., Perez, M. K., Patel, S. C., Gotsdiner, D., Fischbeck, K. H. and Pittman, R. N. (1997). Machado-Joseph disease gene product is a cytoplasmic protein widely expressed in brain. Ann Neurol 41, 453-62.

Peters, M. F., Nucifora, F. C., Jr., Kushi, J., Seaman, H. C., Cooper, J. K., Herring, W. J., Dawson, V. L., Dawson, T. M. and Ross, C. A. (1999). Nuclear targeting of mutant Huntingtin increases toxicity. Mol Cell Neurosci 14, 121-8.

Pichler, A., Gast, A., Seeler, J. S., Dejean, A. and Melchior, F. (2002). The nucleoporin RanBP2 has SUMO1 E3 ligase activity. Cell 108, 109-20.

Pichler, A. and Melchior, F. (2002). Ubiquitin-related modifier SUMO1 and nucleocytoplasmic transport. Traffic 3, 381-7.

Pollitt, S. K., Pallos, J., Shao, J., Desai, U. A., Ma, A. A., Thompson, L. M., Marsh, J. L. and Diamond, M. I. (2003). A Rapid Cellular FRET Assay of Polyglutamine Aggregation Identifies a Novel Inhibitor. Neuron 40, 685-94.

Pountney, D. L., Huang, Y., Burns, R. J., Haan, E., Thompson, P. D., Blumbergs, P. C. and Gai, W. P. (2003). SUMO-1 marks the nuclear inclusions in familial neuronal intranuclear inclusion disease. Exp Neurol 184, 436-46.

Rangasamy, D., Woytek, K., Khan, S. A. and Wilson, V. G. (2000). SUMO-1 modification of bovine papillomavirus E1 protein is required for intranuclear accumulation. J Biol Chem 275, 37999-8004.

Regad, T. and Chelbi-Alix, M. K. (2001). Role and fate of PML nuclear bodies in response to interferon and viral infections. Oncogene 20, 7274-86.

Richon, V. M., Sandhoff, T. W., Rifkind, R. A. and Marks, P. A. (2000). Histone deacetylase inhibitor selectively induces p21WAF1 expression and gene-associated histone acetylation. Proc Natl Acad Sci USA 97, 10014-10019.

Ross, C. A. (2002). Polyglutamine pathogenesis: emergence of unifying mechanisms for Huntington's disease and related disorders. Neuron 35, 819-22.

Ross, C. A., Poirier, M. A., Wanker, E. E. and Amzel, M. (2002). Polyglutamine fibrillogenesis: The pathway unfolds. Proc Natl Acad Sci U S A.

Ross, S., Best, J. L., Zon, L. I. and Gill, G. (2002). SUMO-1 modification represses Sp3 transcriptional activation and modulates its subnuclear localization. Mol Cell 10, 831-42.

Sachdev, S., Bruhn, L., Sieber, H., Pichler, A., Melchior, F. and Grosschedl, R. (2001). PIASy, a nuclear matrix-associated SUMO E3 ligase, represses LEF1 activity by sequestration into nuclear bodies. Genes Dev 15, 3088-103.

Saitoh, H. and Hinchey, J. (2000). Functional heterogeneity of small ubiquitin-related protein modifiers SUMO-1 versus SUMO-2/3. J Biol Chem 275, 6252-8.

Saitoh, H., Pizzi, M. D. and Wang, J. (2002). Perturbation of SUMOlation enzyme Ubc9 by distinct domain within nucleoporin RanBP2/Nup358. J Biol Chem 277, 4755-63.

Salomoni, P. and Pandolfi, P. P. (2002). The role of PML in tumor suppression. Cell 108, 165-70.

Sanchez, I., Mahlke, C. and Yuan, J. (2003). Pivotal role of oligomerization in expanded polyglutamine neurodegenerative disorders. Nature 421, 373-9.

Saudou, F., Finkbeiner, S., Devys, D. and Greenberg, M. (1998). Huntingtin acts in the nucleus to induce apoptosis but death does not correlate with the formation of intranuclear inclusions. Cell 95, 55-66.

Seeler, J. S. and Dejean, A. (2003). Nuclear and Unclear Functions of SUMO. Nature Rev, Mol Cell Biol 4, 690-699.

Seeler, J. S., Marchio, A., Losson, R., Desterro, J. M., Hay, R. T., Chambon, P. and Dejean, A. (2001). Common properties of nuclear body protein SP100 and TIF1alpha chromatin factor: role of SUMO modification. Mol Cell Biol 21, 3314-24.

Shih, H. P., Hales, K. G., Pringle, J. R. and Peifer, M. (2002). Identification of septin-interacting proteins and characterization of the . Smt3/SUMO-conjugation system in Drosophila. J Cell Sci 115, 1259-71.

Shimohata, T., Nakajima, T., Yamada, M., Uchida, C., Onodera, O., Naruse, S., Kimura, T., Koidel, R., Nozakil, K., Sano, Y. et al. (2000). Expanded polyglutamine stretches interact with TAFII130, interfering with CREB-dependent transcription. Nat Gen. 26, 29-36.

Sieradzan, K. A., Mechan, A. O., Jones, L., Wanker, E. E., Nukina, N. and Mann, D. M. (1999). Huntington's disease intranuclear inclusions contain truncated, ubiquitinated huntingtin protein. Exp Neurol 156, 92-9.

Sipione, S., Rigamonti, D., Valenza, M., Zuccato, C., Conti, L., Pritchard, J., Kooperberg, C., Olson, J. M. and Cattaneo, E. (2002). Early transcriptional profiles in huntingtin-inducible striatal cells by microarray analyses. Hum Mol Genet 11, 1953-65.

Skinner, P. J., Koshy, B. T., Cummings, C. J., Klement, I. A., Helin, K., Servadio, A., Zoghbi, H. Y. and Orr, H. T. (1997). Ataxin-1 with an expanded glutamine tract alters nuclear matrix-associated structures. Nature 389, 971-4.

Smith, D. L., Portier, R., Woodman, B., Hockly, E., Mahal, A., Klunk, W. E., Li, X. J., Wanker, E., Murray, K. D. and Bates, G. P. (2001). Inhibition of polyglutamine aggregation in R6/2 HD brain slices-complex dose-response profiles. Neurobiol Dis 8, 1017-26.

Steffan, J. S., Bodai, L., Pallos, J., Poelman, M., McCampbell, A., Apostol, B. L., Kazantsev, A., Schmidt, E., Zhu, Y. Z., Greenwald, M. et al. (2001). Histone deacetylase inhibitors arrest polyglutamine-dependent neurodegeneration in Drosophila. Nature 413, 739-43.

Steffan, J. S., Kazantsev, A., Spasic-Boskovic, O., Greenwald, M., Zhu, Y. Z., Gohler, H., Wanker, E. E., Bates, G. P., Housman, D. E. and Thompson, L. M. (2000). The Huntington's disease protein interacts with p53 and CREB-binding protein and represses transcription. Proc Natl Acad Sci U S A 97, 6763-8.

Steffan, J. S. and Thompson, L. M. (2003). Targeting aggregation in the development of therapeutics for the treatment of Huntington's disease and other polyglutamine repeat diseases. Expert Opin Ther Targets 7, 201-13.

Strudwick, S. and Borden, K. L. (2002). Finding a role for PML in APL pathogenesis: a critical assessment of potential PML activities. Leukemia 16, 1906-17.

Su, H. and Li, S. (2002). Molecular features of human ubiquitin-like SUMO genes and their encoded proteins. Gene 296, 65.

Sugars, K. L. and Rubinsztein, D. C. (2003). Transcriptional abnormalities in Huntington disease. Trends Genet 19, 233-8.

Szebenyi, G., Morfini, G. A., Babcock, A., Gould, M., Selkoe, K., Stenoien, D. L., Young, M., Faber, P. W., MacDonald, M. E., McPhaul, M. J. et al. (2003). Neuropathogenic forms of huntingtin and androgen receptor inhibit fast axonal transport. Neuron 40, 41-52.

Takahashi, J., Fujigasaki, H., Zander, C., El Hachimi, K. H., Stevanin, G., Durr, A., Lebre, A. S., Yvert, G., Trottier, Y., The, H. et al. (2002). Two populations of neuronal intranuclear inclusions in SCA7 differ in size and promyelocytic leukaemia protein content. Brain 125, 1534-43.

Tatham, M. H., Jaffray, E., Vaughan, O. A., Desterro, J. M., Botting, C. H., Naismith, J. H. and Hay, R. T. (2001). Polymeric chains of SUMO-2 and SUMO-3 are conjugated to protein substrates by SAE1/SAE2 and Ubc9. J Biol Chem 276, 35368-74.

Terashima, T., Kawai, H., Fujitani, M., Maeda, K. and Yasuda, H. (2002). SUMO-1 co-localized with mutant atrophin-1 with expanded polyglutamines accelerates intranuclear aggregation and cell death. Neuroreport 13, 2359-64.

Tobin, A. J. and Signer, E. R. (2000). Huntington's disease: the challenge for cell biologists. Trends Cell Biol 10, 531-6.

Trushina, E., Heldebrant, M. P., Perez-Terzic, C. M., Bortolon, R., Kovtun, I. V., Badger, J. D., 2nd, Terzic, A., Estevez, A., Windebank, A. J., Dyer, R. B. et al. (2003). Microtubule destabilization and nuclear entry are sequential steps leading to toxicity in Huntington's disease. Proc Natl Acad Sci U S A 100, 12171-6.

Tsai, Y. C., Fishman, P. S., Thakor, N. V. and Oyler, G. A. (2003). Parkin facilitates the elimination of expanded polyglutamine proteins and leads to preservation of proteasome function. J Biol Chem 278, 22044-22055.

Tsukamoto, T., Hashiguchi, N., Janicki, S. M., Tumbar, T., Belmont, A. S. and Spector, D. L. (2000). Visualization of gene activity in living cells. Nat Cell Biol 2, 871-8.

Ueda, H., Goto, J., Hashida, H., Lin, X., Oyanagi, K., Kawano, H., Zoghbi, H. Y., Kanazawa, I. and Okazawa, H. (2002). Enhanced SUMOylation in polyglutamine diseases. Biochem Biophys Res Commun 293, 307-13.

Verger, A., Perdomo, J. and Crossley, M. (2003). Modification with SUMO. EMBO Rep 4, 137-42.

Verhoef, L. G., Lindsten, K., Masucci, M. G. and Dantuma, N. P. (2002). Aggregate formation inhibits proteasomal degradation of polyglutamine proteins. Hum Mol Genet 11, 2689-700.

Waelter, S., Boeddrich, A., Lurz, R., Scherzinger, E., Lueder, G., Lehrach, H. and Wanker, E. E. (2001). Accumulation of mutant Huntingtin fragments in aggresome-like inclusion bodies as a result of insufficient protein degradation. Mol. Biol. Cell 12, 1393-1407.

Wilson, V. G. and Rangasamy, D. (2001). Intracellular targeting of proteins by sumoylation. Exp Cell Res 271, 57-65.

Wood, J. D., Nucifora, F. C., Duan, K., Zhang, C., Wang, J., Kim, Y., Schilling, G., Sacchi, N., Liu, J. M. and Ross, C. A. (2000). Atrophin-1, the dentato-rubral and pallido-luysian atrophy gene product, interacts with ETO/MTG8 in the nuclear matrix and represses transcription. J. Cell. Biol. 150, 939-948.

Wyttenbach, A., Carmichael, J., Swartz, J., Furlong, R. A., Narain, Y., Rankin, J. and Rubinsztein, D. C. (2000). Effects of heat shock, heat shock protein 40 (HDJ-2) and proteasome inhibition on protein aggregation in cellular models of Huntington's disease. Proc. Natl. Acad. Sci. USA 97, 2898-2903.

Wyttenbach, A., Swartz, J., Kita, H., Thykjaer, T., Carmichael, J., Bradley, J., Brown, R., Maxwell, M., Schapira, A., Orntoft, T. F. et al. (2001). Polyglutamine expansions cause decreased CRE-mediated transcription and early gene expression changes prior to cell death in an inducible cell model of Huntington's disease. Hum. Mol. Gen. 10, 1829-1845.

Yamada, M., Sato, T., Shimohata, T., Hayashi, S., Igarashi, S., Tsuji, S. and Takahashi, H. (2001). Interaction between neuronal intranuclear inclusions and promyelocytic leukemia protein nuclear and coiled bodies in CAG repeat diseases. Am J Pathol 159, 1785-95.

Yamada, M., Wood, J. D., Shimohata, T., Hayashi, S., Tsuji, S., Ross, C. A. and Takahashi, H. (2001). Widespread occurrence of intranuclear atrophin-1 accumulation in the central nervous system neurons of patients with dentatorubral-pallidoluysian atrophy. Ann Neurol 49, 14-23.

Yang, W., Dunlap, J. R., Andrews, R. B. and Wetzel, R. (2002). Aggregated polyglutamine peptides delivered to nuclei are toxic to mammalian cells. Hum Mol Genet 11, 2905-17.

Yasuda, S., Inoue, K., Hirabayashi, M., Higashiyama, H., Yamamoto, Y., Fuyuhiro, H., Komure, O., Tanaka, F., Sobue, G., Tsuchiya, K. et al. (1999). Triggering of neuronal cell death by accumulation of activated SEK1 on nuclear polyglutamine aggregations in PML bodies. Genes Cells 4, 743-56.

Zhong, S., Muller, S., Ronchetti, S., Freemont, P. S., Dejean, A. and Pandolfi, P. P. (2000). Role of SUMO-1-modified PML in nuclear body formation. Blood 95, 2748-52.

Zhong, S., Salomoni, P. and Pandolfi, P. P. (2000). The transcriptional role of PML and the nuclear body. Nat Cell Biol 2, E85-90.

Zoghbi, H. Y. and Orr, H. T. (2000). Glutamine repeats and neurodegeneration. Annu. Rev. Neurosci. 23, 217-247.

Zuccato, C., Ciammola, A., Rigamonti, D., Leavitt, B. R., Goffredo, D., Conti, L., MacDonald, M. E., Friedlander, R. M., Silani, V., Hayden, M. R. et al. (2001). Loss of huntingtin-mediated BDNF gene transcription in Huntington's disease. Science 293, 493-8.

Zuccato, C., Tartari, M., Crotti, A., Goffredo, D., Valenza, M., Conti, L., Cataudella, T., Leavitt, B. R., Hayden, M. R., Timmusk, T. et al. (2003). Huntingtin interacts with REST/NRSF to modulate the transcription of NRSE-controlled neuronal genes. Nat Genet 35, 76-83.

While this invention has been described in detail with reference to a certain preferred embodiments, it should be appreciated that the present invention is not limited to those precise embodiments. Rather, in view of the present disclosure which describes the current best mode for practicing the invention, many modifications and variations would present themselves to those of skill in the art without departing from the scope and spirit of this invention. In particular, it is to be understood that this invention is not limited to the particular methodology, protocols, cell lines, animal species or genera, constructs, and reagents described as such may vary, as will be appreciated by one of skill in the art. The scope of the invention is, therefore, indicated by the following claims rather than by the foregoing description. All changes, modifications, and variations coming within the meaning and range of equivalency of the claims are to be considered within their scope.

We claim:

1. A method of treating neurodegeneration in a patient, comprising
identifying a patient at risk for neurodegeneration; and
administering to the patient a therapeutically effective amount of SUMOylation blocker.

2. The method of claim 1, wherein the SUMOylation blocker is an inhibitor E1 SUMO activating enzyme.

3. The method of claim 1, wherein the SUMOylation blocker is an inhibitor E2 SUMO conjugating enzyme.

4. The method of claim 1, wherein the SUMOylation blocker is an inhibitor E3 SUMO ligating enzyme.

5. The method of claim 4, wherein the inhibitor of E3 SUMO ligating enzyme is a PIAS protein.

6. A method of treating neurodegeneration in a patient, comprising
identifying a patient at risk for neurodegeneration; and
administering to the patient a therapeutically effective amount of deSUMOylation enhancer.

7. The method of claim 6, wherein the deSUMOylation enhancer is SUMO isopeptidase.

8. A method of treating neurodegeneration in a patient, comprising
identifying a patient at risk for neurodegeneration; and
administering to the patient a therapeutically effective amount of a Ubiquitination activator.

9. The method of claim 8, wherein the Ubiquitination activator is an activator of E1 Ubiquitin activating enzyme.

10. The method of claim 8, wherein the Ubiquitination activator is an activator of E1 Ubiquitin conjugating enzyme.

11. The method of claim 8, wherein the Ubiquitination activator is an activator of E3 Ubiquitin ligating enzyme.

12. A method of treating neurodegeneration in a patient, comprising
identifying a patient at risk for neurodegeneration; and
administering to the patient a therapeutically effective amount of deubiquitination inhibitor.

13. The method of claim 12, wherein the deubiquitination inhibitor is an inhibitor of Ubiquitin isopeptidase.

14. A method of treating polyglutamine-expansion-related neurodegeneration in a patient, comprising
identifying a patient at risk for polyglutamine-expansion-related neurodegeneration; and
administering to the patient a therapeutically effective amount of SUMOylation blocker.

15. The method of claim 14, wherein the SUMOylation blocker is an inhibitor E1 SUMO activating enzyme.

16. The method of claim 14, wherein the SUMOylation blocker is an inhibitor E2 SUMO conjugating enzyme.

17. The method of claim 14, wherein the SUMOylation blocker is an inhibitor E3 SUMO ligating enzyme.

18. The method of claim 17, wherein the inhibitor of E3 SUMO ligating enzyme is a PIAS protein.

19. A method of treating polyglutamine-expansion-related neurodegeneration in a patient, comprising
identifying a patient at risk for polyglutamine-expansion-related neurodegeneration; and
administering to the patient a therapeutically effective amount of deSUMOylation enhancer.

20. The method of claim 19, wherein the deSUMOylation enhancer is SUMO isopeptidase.

21. A method of treating polyglutamine-expansion-related neurodegeneration in a patient, comprising
identifying a patient at risk for polyglutamine-expansion-related neurodegeneration; and
administering to the patient a therapeutically effective amount of a Ubiquitination activator.

22. The method of claim 21, wherein the Ubiquitination activator is an activator of E1 Ubiquitin activating enzyme.

23. The method of claim 21, wherein the Ubiquitination activator is an activator of E2 Ubiquitin conjugating enzyme.

24. The method of claim 21, wherein the Ubiquitination activator is an activator of E3 Ubiquitin ligating enzyme.

25. A method of treating polyglutamine-expansion-related neurodegeneration in a patient, comprising
identifying a patient at risk for polyglutamine-expansion-related neurodegeneration; and
administering to the patient a therapeutically effective amount of deubiquitination inhibitor.

26. The method of claim 25, wherein the deubiquitination inhibitor is an inhibitor of Ubiquitin isopeptidase.

27. A method of treating a neurodegenerative disease in a patient, comprising administering to the patient a therapeutically effective amount of SUMOylation blocker.

28. The method of claim 27, wherein the SUMOylation blocker is an inhibitor E1 SUMO activating enzyme.

29. The method of claim 27, wherein the SUMOylation blocker is an inhibitor E2 SUMO conjugating enzyme.

30. The method of claim 27, wherein the SUMOylation blocker is an inhibitor E3 SUMO ligating enzyme.

31. The method of claim 30, wherein the inhibitor of E3 SUMO ligating enzyme is a PIAS protein.

32. The method of claim 27, wherein the neurodegenerative disease is one of more of the group consisting of Huntington's disease, Alzheimer's disease, Parkinson's disease, amyotrophic lateral sclerosis, Kennedy's disease, SCA1, DRPLA, epilepsy, diabetes mellitus, spongiform encephalopathy, prion-related disease, Machado-Joseph's disease and schizophrenia.

33. A method of treating a neurodegenerative disease in a patient, comprising administering to the patient a therapeutically effective amount of deSUMOylation enhancer.

34. The method of claim 33, wherein the deSUMOylation, enhancer is SUMO isopeptidase.

35. The method of claim 33, wherein the neurodegenerative disease is one of more of the group consisting of Huntington's disease, Alzheimer's disease, Parkinson's disease, amyotrophic lateral sclerosis, Kennedy's disease, SCA1, DRPLA, epilepsy, diabetes mellitus, spongiform encephalopathy, prion-related disease, Machado-Joseph's disease and schizophrenia.

36. A method of treating a neurodegenerative disease in a patient, comprising administering to the patient a therapeutically effective amount of a Ubiquitination activator.

37. The method of claim 36, wherein the Ubiquitination activator is an activator of E1 Ubiquitin activating enzyme.

38. The method of claim 36, wherein the Ubiquitination activator is an activator of E1. Ubiquitin conjugating enzyme.

39. The method of claim 36, wherein the Ubiquitination activator is an activator of E3 Ubiquitin ligating enzyme.

40. A method of treating a neurodegenerative disease in a patient, comprising administering to the patient a therapeutically effective amount of a deubiquitination inhibitor.

41. The method of claim 40, wherein the deubiquitination inhibitor is an inhibitor of Ubiquitin isopeptidase.

42. The method of claim 40, wherein the neurodegenerative disease is one of more of the group consisting of Huntington's disease, Alzheimer's disease, Parkinson's disease, amyotrophic lateral sclerosis, Kennedy's disease, SCA1, DRPLA, epilepsy, diabetes mellitus, spongiform encephalopathy, prion-related disease, Machado-Joseph's disease and schizophrenia.

43. A method of treating Huntington's disease in a patient, comprising administering to the patient a therapeutically effective amount of a SUMOylation blocker.

44. A method of treating Huntington's disease in a patient, comprising administering to the patient a therapeutically effective amount of a deSUMOylation enhancer.

45. A method of treating Huntington's disease in a patient, comprising administering to the patient a therapeutically effective amount of a Ubiquitination activator.

46. A method of treating Huntington's disease in a patient, comprising administering to the patient a therapeutically effective amount of a deubiquitination inhibitor.

47. A method of treating Kennedy's disease in a patient, comprising administering to the patient a therapeutically effective amount of a SUMOylation blocker.

48. A method of treating Kennedy's disease in a patient, comprising administering to the patient a therapeutically effective amount of a deSUMOylation enhancer.

49. A method of treating Kennedy's disease in a patient, comprising administering to the patient a therapeutically effective amount of a Ubiquitination activator.

50. A method of treating Kennedy's disease in a patient, comprising administering to the patient a therapeutically effective amount of a deubiquitination inhibitor.

51. A method of treating spinocerebellar ataxia in a patient, comprising administering to the patient a therapeutically effective amount of a SUMOylation blocker.

52. A method of treating spinocerebellar ataxia in a patient, comprising administering to the patient a therapeutically effective amount of a deSUMOylation enhancer.

53. A method of treating spinocerebellar ataxia in a patient, comprising administering to the patient a therapeutically effective amount of a Ubiquitination activator.

54. A method of treating spinocerebellar ataxia in a patient, comprising administering to the patient a therapeutically effective amount of a deubiquitination inhibitor.

55. A method of treating dentatorubral-pallidoluysian atrophy in a patient, comprising administering to the patient a therapeutically effective amount of a SUMOylation blocker.

56. A method of treating dentatorubral-pallidoluysian atrophy in a patient, comprising administering to the patient a therapeutically effective amount of a deSUMOylation enhancer.

57. A method of treating dentatorubral-pallidoluysian atrophy in a patient, comprising administering to the patient a therapeutically effective amount of a Ubiquitination activator.

58. A method of treating dentatorubral-pallidoluysian atrophy in a patient, comprising administering to the patient a therapeutically effective amount of a deUbiquitination inhibitor.

59. A method of treating protein-aggregation-related neurodegeneration in a patient, comprising administering to the patient a therapeutically effective amount of a SUMOylation blocker.

60. A method of treating protein-aggregation-related neurodegeneration in a patient, comprising administering to the patient a therapeutically effective amount of a deSUMOylation enhancer.

61. A method of treating protein-aggregation-related neurodegeneration in a patient, comprising administering to the patient a therapeutically effective amount of a Ubiquitination activator.

62. A method of treating protein-aggregation-related neurodegeneration in a patient, comprising administering to the patient a therapeutically effective amount of a deUbiquitination inhibitor.

63. A method of treating Machado-Joseph's disease in a patient, comprising administering to the patient a therapeutically effective amount of a SUMOylation blocker.

64. A method of treating Machado-Joseph's disease in a patient, comprising administering to the patient a therapeutically effective amount of a deSUMOylation enhancer.

65. A method of treating Machado-Joseph's disease in a patient, comprising administering to the patient a therapeutically effective amount of a Ubiquitination activator.

66. A method of treating Machado-Joseph's disease in a patient, comprising administering to the patient a therapeutically effective amount of a deubiquitination inhibitor.
